(19) United States
(12) Patent Application Publication
(10) Pub. No.: US 2026/0081006 A1
(43) Pub. Date: Mar. 19, 2026

(54) AUTOMATED MONITORING OF OPERATING-ROOM STATISTICS

(71) Applicant: LayerJot, Inc., Menlo Park, CA (US)

(72) Inventors: Etay Gafni, Menlo Park, CA (US); Soren Harner, Palo Alto, CA (US)

(73) Assignee: LayerJot, Inc., Menlo Park, CA (US)

(21) Appl. No.: 19/060,628

(22) Filed: Feb. 21, 2025

Related U.S. Application Data
(60) Provisional application No. 63/694,745, filed on Sep. 13, 2024.

(51) Int. Cl. G16H 40/20; G06V 10/25; G06V 20/52; G16H 10/60; G16H 40/40

(52) U.S. Cl. CPC G16H 40/20; G06V 10/25; G06V 20/52; G16H 10/60; G16H 40/40

(57) ABSTRACT

An electronic device that monitors usage of medical devices (such as a surgical instrument, a surgical screw, surgical supplies or a surgical machine) is described. During at least a medical procedure, the electronic device monitors the use of medical devices in a tray of medical devices or a peel pack of medical devices. Then, the electronic device may provide, addressed to a computer system, a utilization report based at least in part on the monitoring. The utilization report may include historical usage of medical devices: in a type of the medical procedure, for a type of patient and/or by a given medical provider.

### CROSS REFERENCE TO RELATED APPLICATIONS

This application claims priority under 35 U.S.C. 119(e) to U.S. Provisional Application Ser. No. 63/694,745, entitled “Surgical Instrument Use Based on Operating-Room Statistics,” by Etay Gafni, et al., filed on Sep. 13, 2024, the contents of both of which are herein incorporated by reference.

### FIELD

The described embodiments relate to techniques for monitoring usage of medical devices (such as surgical instruments) during a medical procedure based at least in part on historical usage of medical devices: in a type of medical procedure (such as a type of surgery), for a type of patient and/or by a particular surgeon.

### BACKGROUND

Medical devices (such as surgical instruments) are a mainstay of a wide variety of medical procedures. Typically, medical support staff (such as surgical technicians) assemble a set of surgical instruments on a tray for subsequent use in a given medical procedure. Moreover, the medical support staff ensure that the set of surgical instruments and medical devices are sterile.

However, existing approaches for assembling trays are usually human-centric and, thus, prone to error. For example, medical support staff routinely assemble trays in a predefined and fixed manner. Consequently, these trays often include unneeded or unnecessary surgical instruments. The unneeded or unnecessary surgical instruments are not used, which increases waste and cost associated with surgeries.

Moreover, the extra surgical instruments increase the time needed to assemble the trays, and may make errors during tray assembly more likely, such as: including the wrong surgical instrument in a tray, forgetting to include one or more surgical instrument in the tray; insufficient inspection of surgical instruments for damage, bioburden, rust and other issues that can impact patient outcomes (because of assembly-related time pressure); and/or misplacing one or more surgical instruments on the tray.

In turn, the increase in tray-assembly errors and, more generally, in tray complexity can result in delays and associated expense as the errors are corrected. In the worst case, errors during tray assembly can result in additional errors during medical procedures and can adversely impact patient care.

### SUMMARY

In a first group of embodiments, an electronic device that facilitates tray or peel-pack assembly is described. This electronic device includes: an interface circuit that communicates with a computer system; a processor; and memory that stores program instructions, where, when executed by the processor, the program instructions cause the electronic device to perform operations. Notably, during operation, the electronic device receives or obtains, from the computer system, information specifying medical devices to include in the tray of medical devices or the peel pack of medical devices based at least in part on historical usage of medical devices: in a type of medical procedure, for a type of patient and/or by a given medical provider (such as a surgeon). Then, the electronic device provides (e.g., on a display associated with the electronic device), the information and/or an instruction to assemble the tray of medical devices or the peel pack of medical devices based at least in part on the information.

Note that the medical devices may include: a surgical instrument, a surgical screw, surgical supplies and/or a surgical machine. In some embodiments, the information may include: a type of a medical device in the medical devices; a classification of the medical device; an identification code of the medical device (such as a SKU); membership of the medical device in a group (such as a surgical tray, peel pack or set); or a manufacturer of the medical device.

Moreover, the medical procedure may include a type of surgery.

Furthermore, the type of patient may have a type of medical condition (such as a disease) and/or a severity of the type of medical condition.

Additionally, a number of the medical devices included in the information for use in the tray of medical devices or the peel pack of medical devices may be reduced relative to a second number of medical devices in a predefined set of medical devices.

In some embodiments, the operations include automatically assembling (e.g., using a robot) the tray of medical devices or the peel pack of medical devices based at least in part on the information. For example, automatically assembling the tray of medical devices or the peel pack of medical devices may include: identifying a medical device based at least in part on the inclusion of the medical device in the information; selecting the medical device; and placing the medical device at a given location and orientation on the tray of medical devices or in the peel pack of medical devices. Note that the electronic device may include an image sensor that acquires one or more images, and the electronic device may identify the medical device based at least in part on the one or more images. Moreover, the medical device may be identified based at least in part on a visual attribute of the medical device, such as: a shape of at least a portion of the medical device (e.g., a predefined or predetermined shape of at least the portion of the medical device, which may be included in a data structure that is accessed during the medical procedure), a texture of at least a second portion of the medical device (e.g., a predefined or predetermined texture of at least the portion of the medical device, which may be included in a data structure that is accessed during the medical procedure); and/or a reflectivity of at least a third portion of the medical device (e.g., a predefined or predetermined reflectivity of at least the portion of the medical device, which may be included in a data structure that is accessed during the medical procedure). However, the disclosed assembly techniques are not limited to descriptors such as shape, texture and/or reflectivity. More generally, the assembly techniques may use embedding of multiple descriptors, such as 512 descriptors.

The identifying may be performed by the electronic device. Alternatively or additionally, the electronic device may: provide the one or more images to the computer system; and receive the identification of the medical device from the computer system.

Note that the information may include: a barcode of a first medical device in the medical devices; a SKU of a second medical device in the medical devices; and/or visual attributes of a third medical device in the medical devices.

Prior to the identifying, the electronic device may: scale a size of an image in the one or more images; and rotate the scaled image to maximize second information associated with pixels in a barcode or text associated with the medical device. When the information includes a barcode, the electronic device may assess quality of a first image in the one or more images and, based at least in part on the assessed quality, may selectively adjust a zoom of the image sensor.

Moreover, the information may include a radio-frequency (RF) identifier of a fourth medical device in the medical devices. The electronic device may identify the fourth medical device based at least in part on the RF identifier.

In some embodiments, the information for a given medical device in the medical devices may be associated with multiple locations on the given medical device.

Another embodiment provides the computer system.

Another embodiment provides a computer-readable storage medium that stores program instructions for use with the electronic device or the computer system. When executed by the electronic device or the computer system, the program instructions cause the electronic device or the computer system to perform at least some of the aforementioned operations.

Another embodiment provides a method, which may be performed by the electronic device or the computer system. This method includes at least some of the aforementioned operations.

In a second group of embodiments, an electronic device that monitors usage of medical devices is described. This electronic device includes: an interface circuit that communicates with a computer system; an image sensor that acquires one or more images; a processor; and memory that stores program instructions, where, when executed by the processor, the program instructions cause the electronic device to perform operations. Notably, during operation, the electronic device monitors use of medical devices in a tray of medical devices or a peel pack of medical devices during at least a medical procedure. Then, the electronic device provides, addressed to the computer system, a utilization report based at least in part on the monitoring, where the utilization report includes historical usage of medical devices: in a type of the medical procedure, for a type of patient and/or by a given medical provider (such as a surgeon).

In some embodiments, the electronic device may provide a recommendation for one or more medical devices to be included in another instance of the tray of medical devices or the peel pack of medical devices for use in a future medical procedure, where the recommendation is based at least in part on the historical usage of medical devices: in the type of the medical procedure, for the type of patient and/or by the given medical provider.

Note that the recommendation may be based at least in part on a preference of the given medical provider.

Moreover, the monitoring may include monitoring of a Mayo stand or a table holding at least the tray of medical devices or the peel pack of medical devices during the medical procedure.

Furthermore, the electronic device may predict a medical device will be needed in the medical procedure, where the medical device is not included in the tray of medical devices or the peel pack of medical devices. Then, the electronic device may provide an instruction to obtain the medical device or may automatically obtain the medical device.

In some embodiments, the recommendation is provided to the computer system, which may implement electronic medical record (EMR) software.

Note that the medical devices may include: a surgical instrument, a surgical screw, surgical supplies and/or a surgical machine. In some embodiments, the monitoring may include determining: a type of a medical device in the medical devices; a classification of the medical device; an identification code of the medical device (such as a SKU); membership of the medical device in a group (such as a surgical tray, peel pack or set); or a manufacturer of the medical device.

Moreover, the medical procedure may include a type of surgery.

Furthermore, the type of patient may have a type of medical condition (such as a disease) and/or a severity of the type of medical condition.

Additionally, the electronic device may document a particular or discrete moment in the medical procedure. Note that the documentation may be based at least in part by a trigger, such as: motion of a gloved hand; an absence of the gloved hand in a field of view of the image sensor; placement of a medical device in a predefined portion in a frame of the image sensor; etc. In general, the trigger may be explicit or implicit. Alternatively, the electronic device may perform continuous monitoring during the medical procedure.

In some embodiments, the electronic device may redact information acquired during the monitoring, such as protected health information associated with a patient or an identity of the medical provider.

Moreover, during the monitoring, the electronic device may provide an instruction to change a perspective (such as an orientation or a field of view) of the image sensor during the medical procedure. This instruction may ensure that areas of interest (such as an edge of a towel, a table holding at least the tray of medical devices or the peel pack of medical devices, etc.) is included in a field of view of the image sensor during the monitoring.

Furthermore, the monitoring may involve: identifying the medical devices observed during the monitoring; and/or verifying the medical devices observed during the monitoring.

Another embodiment provides the computer system.

Another embodiment provides a computer-readable storage medium that stores program instructions for use with the electronic device or the computer system. When executed by the electronic device or the computer system, the program instructions cause the electronic device or the computer system to perform at least some of the aforementioned operations.

Another embodiment provides a method, which may be performed by the electronic device or the computer system. This method includes at least some of the aforementioned operations.

This Summary is provided for purposes of illustrating some exemplary embodiments, so as to provide a basic understanding of some aspects of the subject matter described herein. Accordingly, it will be appreciated that the above-described features are examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a block diagram illustrating an example of communication among an electronic device and a computer system in accordance with an embodiment of the present disclosure.

FIG. 2 is a flow diagram illustrating an example of a method for identifying a medical device using an electronic device in FIG. 1 in accordance with an embodiment of the present disclosure.

FIG. 3 is a drawing illustrating an example of communication among an electronic device and a computer system in FIG. 1 in accordance with an embodiment of the present disclosure.

FIG. 4 is a drawing illustrating an example of a medical device in accordance with an embodiment of the present disclosure.

FIG. 5 is a drawing illustrating an example of a medical device with an obscured portion in accordance with an embodiment of the present disclosure.

FIG. 6 is a flow diagram illustrating an example of a method for facilitating assembly of a tray of medical devices or a peel pack of medical devices using an electronic device in FIG. 1 in accordance with an embodiment of the present disclosure.

FIG. 7 is a drawing illustrating an example of communication between an electronic device and a computer system in FIG. 1 in accordance with an embodiment of the present disclosure.

FIG. 8 is a flow diagram illustrating an example of a method for monitoring usage of medical devices during a medical procedure using an electronic device in FIG. 1 in accordance with an embodiment of the present disclosure.

FIG. 9 is a drawing illustrating an example of communication between an electronic device and a computer system in FIG. 1 in accordance with an embodiment of the present disclosure.

FIG. 10 is a drawing illustrating an example of a flow associated with a surgical procedure.

FIG. 11 is a drawing illustrating an example of a flow associated with the surgical procedure of FIG. 10.

FIG. 12 is a drawing illustrating an example of a flow associated with the surgical procedure of FIGS. 10 and 11.

FIG. 13 is a drawing illustrating an example of a flow associated with a surgical procedure in accordance with an embodiment of the present disclosure.

FIG. 14 is a drawing illustrating an example of a user interface in accordance with an embodiment of the present disclosure.

FIG. 15 is a drawing illustrating an example of a user interface in accordance with an embodiment of the present disclosure.

FIG. 16 is a drawing illustrating an example of a user interface in accordance with an embodiment of the present disclosure.

FIG. 17 is a drawing illustrating an example of a user interface in accordance with an embodiment of the present disclosure.

FIG. 18 is a drawing illustrating an example of a user interface in accordance with an embodiment of the present disclosure.

FIG. 19 is a drawing illustrating an example of a user interface in accordance with an embodiment of the present disclosure.

FIG. 20 is a drawing illustrating an example of a user interface in accordance with an embodiment of the present disclosure.

FIG. 21 is a drawing illustrating an example of a user interface in accordance with an embodiment of the present disclosure.

FIG. 22 is a drawing illustrating an example of an image of a tray of medical devices during a surgical procedure in accordance with an embodiment of the present disclosure.

FIG. 23 is a drawing illustrating an example of a user interface in accordance with an embodiment of the present disclosure.

FIG. 24 is a drawing illustrating an example of a user interface in accordance with an embodiment of the present disclosure.

FIG. 25 is a drawing illustrating an example of a user interface in accordance with an embodiment of the present disclosure.

FIG. 26 is a drawing illustrating an example of a user interface in accordance with an embodiment of the present disclosure.

FIG. 27 is a drawing illustrating an example of a user interface in accordance with an embodiment of the present disclosure.

FIG. 28 is a drawing illustrating an example of a user interface in accordance with an embodiment of the present disclosure.

FIG. 29 is a drawing illustrating an example of a user interface in accordance with an embodiment of the present disclosure.

FIG. 30 is a block diagram illustrating an electronic device in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

In a first group of embodiments, an electronic device that facilitates assembly of a tray of medical devices (such as a surgical instrument, a surgical screw, surgical supplies or a surgical machine) or a peel pack of medical devices (which is sometimes referred to as a ‘sterilization pouch’). During operation, the electronic device may receive or obtain, from a computer system, information specifying medical devices to include in a tray of medical devices or a peel pack of medical devices based at least in part on historical usage of medical devices: in a type of medical procedure, for a type of patient and/or by a given medical provider. Then, the electronic device may provide an instruction to assemble the tray of medical devices or the peel pack of medical devices based at least in part on the information. Alternatively, the electronic device (or an associated robot) may automatically assemble the tray of medical devices or the peel pack of medical devices based at least in part on the information.

In a second group of embodiments, an electronic device that monitors usage of medical devices (such as a surgical instrument, a surgical screw, surgical supplies or a surgical machine) is described. During at least a medical procedure, the electronic device monitors the use of medical devices in a tray of medical devices or a peel pack of medical devices. Then, the electronic device may provide, addressed to a computer system, a utilization report based at least in part on the monitoring. The utilization report may include historical usage of medical devices: in a type of the medical procedure, for a type of patient and/or by a given medical provider.

By performing these operations, these monitoring and/or assembly techniques may allow the electronic device to accurately and efficiently facilitate the assembly of the tray of medical devices or the peel pack of medical devices. Moreover, the monitoring and/or assembly techniques may facilitate improved workflows. For example, the monitoring and/or assembly techniques may facilitate tray or peel-pack assembly based at least in part on data-centric monitoring of medical procedures, such as surgeries. This dynamic approach may reduce waste (such as the inclusion of unneeded or unnecessary medical devices in a tray or a peel pack), assembly time and cost, and may simplify trays or peel packs (e.g., by reducing the number of medical devices included in a tray or a peel pack). Moreover, the monitoring and/or assembly techniques may reduce errors, both during the assembly of trays or peel packs (such as including the wrong medical devices in a tray or a peel pack, forgetting to include one or more medical devices in the tray or the peel pack, and/or misplacing one or more medical devices on the tray or in the peel pack), as well as in medical procedures that use medical devices included in a tray or a peel pack. Therefore, the monitoring and/or assembly techniques may improve patient care and may improve satisfaction of a user, such as a surgical technician or medical support staff.

In the discussion that follows, one or more electronic devices communicate packets or frames in accordance with a wireless communication protocol, such as: a wireless communication protocol that is compatible with an IEEE 802.11 standard (which is sometimes referred to as ‘Wi-Fi®,’ from the Wi-Fi Alliance of Austin, Texas), Bluetooth, a cellular-telephone network or data network communication protocol (such as a third generation or 3G communication protocol, a fourth generation or 4G communication protocol, e.g., Long Term Evolution or LTE (from the 3rd Generation Partnership Project of Sophia Antipolis, Valbonne, France), LTE Advanced or LTE-A, a fifth generation or 5G communication protocol, or other present or future developed advanced cellular communication protocol), and/or another type of wireless interface (such as another wireless-local-area-network interface). For example, an IEEE 802.11 standard may include one or more of: IEEE 802.11a, IEEE 802.11b, IEEE 802.11g, IEEE 802.11-2007, IEEE 802.11n, IEEE 802.11-2012, IEEE 802.11-2016, IEEE 802.11ac, IEEE 802.11ax, IEEE 802.11ba, IEEE 802.11be, IEEE 802.11bn or other present or future developed IEEE 802.11 technologies. Moreover, an access point, a radio node or a base station in a network may communicate with a local or remotely located computer using a wired communication protocol, such as a wired communication protocol that is compatible with an IEEE 802.3 standard (which is sometimes referred to as ‘Ethernet’), e.g., an Ethernet II standard. However, a wide variety of communication protocols may be used, including wired and/or wireless communication. In the discussion that follows, Wi-Fi or a cellular-telephone communication protocol, and Ethernet are used as illustrative examples.

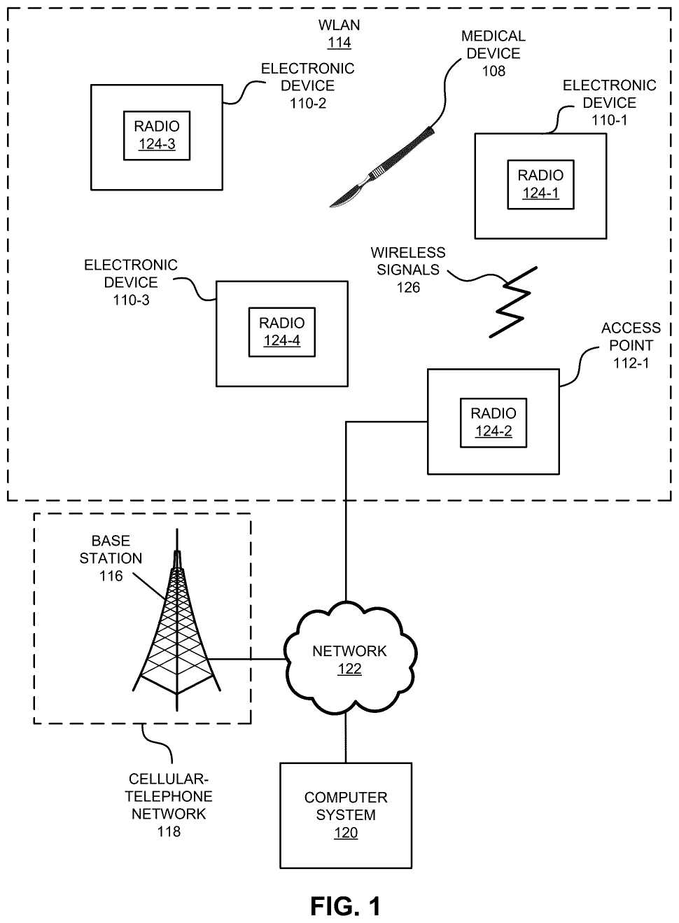

FIG. 1 presents a block diagram illustrating an example of communication among one or more electronic devices 110 (such as a cellular telephone), an access point 112 in a wireless local area network (WLAN) 114, a base station 116 in a cellular-telephone network 118, and a computer system 120 (or a group of one or more computers). Notably, electronic devices 110 may communicate with access point 112 and/or base station 116 using wireless communication. Moreover, access point 112 and/or base station 116 may provide access to a network 122 (such as the Internet, a cable network, etc.) that is external to WLAN 114 or cellular-telephone network 114. Note that access point 112 may include a physical access point and/or a virtual access point that is implemented in software that executes in an operating system of an electronic device or a computer.

Access point 112 may communicate with network 122 and/or base station 116 may communicate with cellular-telephone network 118 and/or network 122 using wired communication and/or wireless communication. This wired or wireless communication may occur via an intra-net, a mesh network, point-to-point connections and/or the Internet and may use a network communication protocol, such as Ethernet. This network may include one or more routers and/or switches (not shown). Furthermore, the wireless communication using Wi-Fi may involve: transmitting advertising frames on wireless channels, detecting one another by scanning wireless channels, establishing connections (for example, by transmitting association or attach requests), and/or transmitting and receiving packets or frames (which may include the association requests and/or additional information as payloads). In some embodiments, the wired and/or wireless communication with access point 112 also involves the use of dedicated connections, such as via a peer-to-peer (P2P) communication technique.

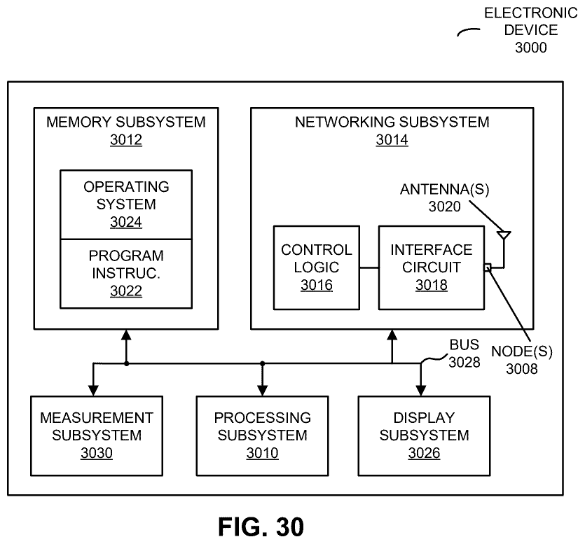

As described further below with reference to FIG. 30, electronic devices 110, access point 112, base station 116 and/or computer system 120 may include subsystems, such as a networking subsystem, a memory subsystem and a processor subsystem. In addition, electronic devices 110, access point 112 and/or base station 116 may include radios 124 in the networking subsystems. More generally, electronic devices 110 and access point 112 can include (or can be included within) any electronic devices with the networking subsystems that enable electronic devices 110 and access point 112 to communicate using wireless and/or wired communication. This wireless communication can include transmitting advertisements on wireless channels to enable electronic devices 110 and access point 112 to make initial contact or detect each other, followed by exchanging subsequent data/management packets or frames (such as association requests and responses) to establish a connection, configure security options (e.g., Internet Protocol Security), transmit and receive packets or frames via the connection, etc. Note that while instances of radios 124 are shown in electronic devices 110, access point 112 and base station 116, one or more of these instances may be different from the other instances of radios 124.

As can be seen in FIG. 1, wireless signals 126 (represented by a jagged line) are transmitted from radio 124-1 in electronic device 110-1. These wireless signals may be received by radio 124-2 in access point 112. Notably, electronic device 110-1 may transmit packets or frames. In turn, these packets or frames may be received by access point 112. Moreover, access point 112 may allow electronic device 110-1 to communicate with other electronic devices, computers and/or servers via network 122.

Note that the communication among electronic devices 110, access point 112 and/or base station 116 may be characterized by a variety of performance metrics (which are sometimes referred to as ‘communication performance metrics’), such as: a received signal strength (RSSI), a data rate, a data rate for successful communication (which is sometimes referred to as a ‘throughput’), an error rate (such as a retry or resend rate), a mean-square error of equalized signals relative to an equalization target, intersymbol interference, multipath interference, a signal-to-noise ratio (SNR), a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’).

In the described embodiments, processing a packet or frame in electronic devices 110, access point 112, and/or base station 116 may include: receiving signals (such as wireless signals 126) with the packet or frame; decoding/extracting the packet or frame from received wireless signals 126 to acquire the packet or frame; and processing the packet or frame to determine information contained in the packet or frame.

Although we describe the network environment shown in FIG. 1 as an example, in alternative embodiments, different numbers or types of electronic devices may be present. For example, some embodiments comprise more or fewer electronic devices. As another example, in another embodiment, different electronic devices are transmitting and/or receiving packets or frames.

We now describe embodiments of identification techniques. Human identification of a medical device can result in errors, additional complexity and/or expense. Moreover, identification of the medical device may be complicated by at least partial obscuring of the medical device.

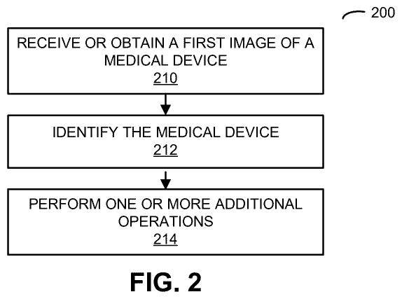

As described further below with reference to FIGS. 2-6, in order to address these problems an electronic device (such as electronic device 110-1) may perform the identification techniques for the identification (such as at least partially automated identification) of a medical device 108. Note that medical device 108 may include: a surgical instrument, a surgical screw, surgical supplies and/or a surgical machine.

During the identification techniques, electronic device 110-1 may execute program instructions or software that performs one or more operations. Note that the program instructions may be a standalone executable that is installed on electronic device 110-1 and executed in an environment of electronic device 110-10 (such as by an operating system on electronic device 110-1). Alternatively or additionally, program instructions may be executed in the environment of a Web browser, such as: a Web-browser plugin, a Web application, a native application leveraging one or more application programming interfaces, and/or a standalone embedded application. In some embodiments, at least a portion of the functionality associated with the identification techniques is implemented using a client-server architecture, e.g., by computer system 120 via WLAN 114, cellular-telephone network 116 and/or network 122). Note that the program instructions may include configuration instructions for a preinstalled augmented reality application (such as a neural network or supervised-learning model) or container on electronic device 110-1. These configuration instructions may be provided to electronic device 110-1, and may tailor or customize the preinstalled augmented reality application or container, so that, when executed, it performs the operations associated with the augmented reality application.

Notably, during the identification techniques, electronic device 110-1 may receive or obtain one or more images of medical device 108, where at least a portion of medical device 108 is partly obscured by an object in the first image or is damaged or degraded (such as a scratch). For example, electronic device 110-1 may acquire the one or more images. Alternatively or additionally, electronic device 110-1 may access one or more previously determined images in memory or may receive the one or more images from another electronic device. Note that the object may include a portion of a human hand or another medical device.

Then, electronic device 110-1 may identify medical device 108 based at least in part on information associated with a second portion of medical device 108 that is not partially obscured by the object or that is not damaged or degraded, where the information includes a visual attribute of medical device 108. Note that the information may include: a barcode of medical device 108, a SKU (or catalog number, identification code or name) of medical device 108, a shape of at least the second portion of medical device 108, a texture of medical device 108; and/or a reflectivity of medical device 108. In some embodiments, electronic device 110-1 may determine an RF identifier of medical device 108. In some embodiments, electronic device 110-1 may determine the weight of medical device 108, which may be used to facilitate identification of medical device 108. Electronic device 108 may identify medical device 108 based at least in part on the RF identifier. The identification of medical device 108 may include: a type of medical device 108; a classification of medical device 108; membership of the medical device 108 in the tray or set; and/or a manufacturer of medical device 108. More generally, the identification techniques may use one or more descriptors or features (such as a property link ring handle, quantized dimensions of the medical device, etc.) to identify at least a subset of the medical devices.

The one or more images may be acquired using one or more sensors in electronic device 110-1, such as one or more image sensors (e.g., a CCD or a CMOS image sensor). Electronic device 110-1 may acquire the one or more images when at least medical device 108 is within a field of view of the one or more image sensors. More generally, electronic device 110-1 may perform one or more measurements using: one or more wireless sensors (such as an interface circuit and an antenna), one or more time-of-flight sensors, one or more radar sensors, one or more ultrasound sensors, a scale to measure weight, and/or another type of non-invasive or non-contact measurement sensor. Note that the one or more measurements may include transmitting and/or receiving signals. For example, the one or more measurements may include an RF identifier of medical device 108.

In some embodiments, the one or more images may include: a single image, video (or a temporal or a spatial sequence of images), complex information (phase and amplitude), depth information (such as a depth image), color (according to a color space, such as RGB, a color space extending outside the visual spectrum, etc.), an amount or an intensity of light (such as from a light meter), information in one or more bands of frequencies or wavelengths, such as: an infrared band, a visible band, an ultraviolet band, etc.

Moreover, medical device 108 in the one or more images may be identified using image analysis. For example, medical device 108 may be identified using a neural network (such as convolutional neural network, a vision transformer or a large language model) and/or a trained machine-learning model (such as a supervised-learning model or an unsupervised-learning model, e.g., support vector machines, classification and regression trees, logistic regression, LASSO, linear regression and/or another linear or nonlinear machine-learning model). Moreover, the machine-learning model may include one or more of: an edge or a line-segment detector, a texture-based feature detector, a texture-less feature detector, a scale invariant feature transform (SIFT)-like object-detector, a speed-up robust-features (SURF) detector, a binary-descriptor (such as ORB) detector, a binary robust invariant scalable keypoints (BRISK) detector, a fast retinal keypoint (FREAK) detector, a binary robust independent elementary features (BRIEF) detector, a features from accelerated segment test (FAST) detector, a motion detector (such as a Gaussian-mixture model) etc. In some embodiments, relative positions in the scene determined through scene analysis and object tracking may be used. Techniques for e scene analysis may include photogrammetry to obtain measurements from images. Note that real-time scene analysis with tracking may involve a Kalman or Bayes filter technique, which may build a state model over previous frames, such that the classification and analysis of one or more medical devices in a scene or the field of view is updated over multiple images in a sequence. Simultaneous localization and mapping (SLAM) may be used to localize one or more image sensors in the world in real time and to provide a frame of reference to describe components of a medical device or set of medical devices. Thus, in the identification techniques, photogrammetry, localization, mapping, and tracking may be generalized and combined with classification-based methods using, e.g., Bayesian inference. In these embodiments, the identification techniques may use: an inertial measurement (e.g., from an accelerometer and/or a gyroscope) to help determine the scale of one or more medical devices; and/or a light sensor to determine an illumination level to assist with light-balances or to determine a color or a type of material.

Moreover, the image analysis may be performed locally on electronic device 110-1 (e.g., electronic device may identify medical device 108) and/or remotely by computer system 120 based on communication via network 122 (e.g., electronic device 110-1 may provide an image to computer system 120 and may receive, from the computer system 120, information that specifies or identified medical device 108).

In some embodiments, medical device 108 is identified using a predictive model (such as a supervised machine-learning model or a neural network) that performs classification (e.g., what type of medical device is medical device 108). For example, the predictive model may include You Only Look Once or Yolo (from the University of Washington, Seattle, Washington). Alternatively or additionally, the identification operation may be performed using a search technique. For example, a user may provide at least an image of medical device 108 and the identification may involve a similarity match with a corpus or a dataset of information associated with medical devices. The image of medical device 108 may be analyzed (e.g., using image analysis) to create a condensed or semantically meaningful set of features associated with medical device 108, which are then compared to the corpus of the dataset to identify one or more potential matches. Notably, a fine-tuned neural network, which was trained with triplet loss, may analyze the image to provide a vector of numerical values for different features (such as 512 features) that represent an overall appearance of medical device 108 (which is sometimes referred to as an ‘embedding vector’). In addition, one or more image-analysis or image-processing techniques may be used to extract additional features associated with medical device 108, including one or more of: a true or absolute size of medical device 108, classification of a tip of medical device 108, identification of one or more loops in medical device 108, information that specifies a topology of medical device 108, one or more image moments of medical device 108, an area of medical device 108, at least a portion of a shape of medical device 108 and/or another type of feature. In some embodiments, the neural network is applied to one or more sub-portions of the image to generate one or more additional vectors of embedded features. Then, a dimensional reduction technique may be applied to the vector, the one or more additional vectors and/or the extracted features. For example, the dimensional reduction technique may include: principle component analysis (PCA), singular value decomposition (SVD), t-distributed stochastic neighbor embedding or t-SNE (in which the relative distance between features is maintained in a lower-dimensional space as in a higher-dimensional space), and/or another dimensional reduction technique.

For example, given an image of a medical device and a detailed description, a vision-language model, such as Contrastive Language-Image Pre-Training or CLIP (from OpenAI of San Francisco, California), may be trained to align the image and the text in a joint embedding space. Then, a medical instrument may be identified by matching an embedding of an image against a database or data structure of embeddings derived from descriptions. From the joint embedding space, a generative neural network (e.g. a Flamingo decoder) may be applied to generate a textual description of the medical device, including any visual attributes. The text description and/or visual attribute may be communicated to the user. Alternatively, one or more visual attributes may remain latent semantic concepts in the embedding. Next, a search over similar representations of medical devices in the corpus of the dataset is performed to identify potential matches. For example, the search may use one or more of nearest neighbor or approximate-nearest techniques, such as cosine similarity (or an inner dot product), a weighted summation of Euclidean distance, locality sensitive hashing, Hierarchical Navigable Small World graphs, etc. This embedding search may be performed via matrix multiplication or by searching an embedding data structure supporting nearest-neighbor queries. When multiple potential matches are identified, a probabilistic comparison of distribution-based features of the potential matches with the information associated with the image may be performed to identify medical device 108. In some embodiments, given the top potential matches, the electronic device may employ a re-ranking technique to score each match against all available information. The re-ranking technique may perform one or more of: geometric verification, Bayesian inference, logical inference, etc.

Note that the identification process may provide speed, accuracy and scale. For example, the identification process may be able to identify matches from a corpus or a dataset of 30,000-100,000 medical devices or instruments. In some embodiments, a new object (such as a new medical device) may be added to the corpus or the dataset by providing 10 images of the new object (e.g., the 10 images may provide sufficient information for the new object to be rapidly and accurate identified in the future using the aforementioned identification process or techniques). In other embodiments, a new object (such as a medical device) may be added to the corpus or the dataset by providing a single textual description of its properties and attributes (e.g. an embedded query image may be matched uniquely against the embedded description in the vision-language joint embedding space).

Moreover, after medical device 108 is identified, electronic device 110-1 may display or provide classification information and/or metadata associated with medical device 108. Notably, the classification information and/or the metadata associated with medical device 108 may include: a name (e.g., of a surgical instrument or a tool), a type of medical device, a classification of medical device 108, a manufacturer of medical device 108, a category, a color, a material, heads or tails, a denomination or numerical value, a relative measurement or dimension (or an absolute measurement or dimension if scale is specified or recovered), a shape, a topological relationship (e.g., a locked padlock), a geometric arrangement, an open or closed state (such as an off-state for a switch), an ordering, etc. For example, a name or category may include a surgical instrument; a shape may include that of a scalpel; a color temperature; a material may include metal, plastic, wood, etc.; a state such as open or closed for scissors or clamps; a geometric arrangement of objects and orders may include a sequence of surgical instruments in a set of surgical instruments; a sequence of objects from left to right; subcomponents of an object, such as the blades or handles of scissors; an RF identifier; the results of operations such as finding objects, counting objects, localizing the position of an object in a three-dimensional (3D) coordinate system, etc. Note that classification information may be derived from metadata stored in a database or data structure by identifying the exact record of the instrument or it may be derived by applying a generative neural-network decoder to extract attributes from an embedding of visual attributes. The geometric arrangements may be determined by detecting and segmenting medical devices in the scene and then applying scene analysis (such as photogrammetry) to determine measurements and spatial relationships. Given a reference object in the scene (such as a ruler or an object having a known size), the electronic device may recover scale in the image and may present measurements to a unit of measure. For example, this can be done by estimating a projective transformation between the image and the world based on the reference object.

When medical device 108 cannot be uniquely identified, electronic device 110-1 may display one or more queries or questions for classification information and/or metadata associated with potential matches for medical device 108. In response, the user may provide feedback, such as the classification information and/or the metadata for medical device 108 to electronic device 110-1. For example, the user may provide the classifications using a user interface (such as a keyboard, a touch pad, a touch-sensitive display, another human-interface device, etc.) and/or a voice-recognition user interface. In some embodiments, the user may provide inputs to electronic device 110-1 during at least a portion of the identification techniques using a human-electronic device interface.

In some embodiments, electronic device 110-1 may provide or display a classification for medical device 108 (such as using a set of predefined or predetermined classifications, e.g., classifications that electronic device 110-1 and/or computer system 120 can recognize). Note that electronic device 110-1 and/or computer system 120 may determine the classification(s) using the same or a second neural network and/or machine-learning model (such as a supervised-learning model or an unsupervised-learning model). In some embodiments, medical device 108 may be identified (and a classification may be specified) using RF identification, a barcode, a Quick Response (QR) code, a fiduciary markers, text or logos on packaging, etc.

In general, information acquired about medical device 108 (such as from the one or more images) may be analyzed or assessed by electronic device 110-1 and/or computer system 120 using one or more scene analysis models in order to tune and optimize a scene-analysis model to characteristics of electronic device 110-1, such as the target hardware. This may include training smaller models for less powerful hardware, quantizing models, pruning models, etc., depending on the type of electronic device and its capabilities (such as whether the one or more images sensors are capable of acquiring 3D or depth information, images outside of the visible band of frequencies, e.g., in an infrared band of frequencies, etc.).

Moreover, one or more inspection criteria associated with at least medical device 108 may be used by electronic device 110-1 and/or computer system 120 to analyze or assess medical device 108. In some embodiments, the user may have previously provided or specified the one or more inspection criteria to electronic device 110-1 and/or computer system 120. For example, one or more inspection criteria may include damage or degradation, such as a scratch, or the presence of obscuring substance, such as tape. While the damage or degradation may be used in the identification, in some embodiments a remedial action may be performed when this occurs, such as providing an instruction to a flip the medical device or instrument over so that another side may be used in the identification. Alternatively, in some embodiments, electronic device 110-1 and/or computer system 120 may determine the one or more inspection criterion based at least in part on analysis of a context (or visual context) of at least a portion of medical device 108 in the one or more images. Note that the user may approve or modify (such as provide a revision to) the determined one or more inspection criteria. In general, the analysis of the context, and thus the determination of the one or more inspection criteria, may be performed locally on electronic device 110-1 and/or remotely by computer system 120 based at least in part on communication via network 122. Furthermore, electronic device 110-1 (and/or computer system 120) may determine the one or more inspection criteria and/or may perform the analysis of the context using the same or a third neural network and/or machine-learning model (such as a supervised-learning model or an unsupervised-learning model).

In some embodiments, the one or more inspection criteria may be determined based at least in part on questions associated with at least the subset of medical device 108 that are provided (e.g., displayed) by electronic device 110-1 to the user, and answers associated with the one or more questions that are received from the user. Note that the received answers may include a revision to at least one of the one or more inspection criteria that are determined by electronic device 110-1 and/or computer system 120. For example, electronic device 110-1 may perform natural language processing and semantic parsing (and, more generally, semantic reasoning) to determine the one or more inspection criteria from the answers. Alternatively, as noted previously, even in embodiments where electronic device 110-1 does not provide questions, electronic device 110-1 may receive a revision from the user to at least one of the one or more inspection criteria, which may have been determined by electronic device 110-1 and/or computer system 120.

Note that the one or more inspection criteria may correspond to one or more attributes or characteristics of one or more medical devices, which may correspond to the context. For example, the one or more attributes or the context may include one or more of: a spatial arrangement (or intra-relationships or interrelationships, e.g., between objects in an image or within a medical device) of the objects in the one or more medical devices, an order of the one or more medical devices, a pattern corresponding to the one or more medical devices, a number of the one or more medical devices, one or more numerical values corresponding to the one or more medical devices, an orientation of the one or more medical devices, a material of the one or more medical devices (such as plastic or metal), a shape of the one or more medical devices (such as a ball, a sphere, a cube, etc.), a value of or associated with the one or more medical devices, measurements relative to a physical or a virtual coordinate system, a temporal or a spatial relationship among the one or more medical devices, or states or actions associated with the one or more medical devices (such as a clean or dirty, open or closed, etc.). Thus, the one or more attributes may include a color and/or a number of the one or more medical devices, and the one or more inspection criteria may be, e.g., that “a medical device is clean.” More generally, the one or more inspection criteria may include business logic to apply to a given image, such as: patterns, colors, size, a shaped inspection region (e.g., a line, a box, an l-shaped region, etc.), a value (such as a numerical value, barcode-side up or barcode-side down (and, more generally, a top side or a bottom side), a number on the surface of a medical device, etc.), a scratch, damage, contamination, etc. For example, the electronic device may identify a medical device that includes a drill set with a drill motor and five bits, each with a colored label. (These components of the drill set may belong together and may share one or more common properties.) After identifying the drill motor by barcode, the electronic device may apply an inspection criterion that counts the components and verifies the colors on the labels. Notably, an inspection criterion may determine the presence of tape on the medical device that would interfere with accurate identification. In some embodiments, when this occurs, an instruction for a remedial action may be provided, such as an instruction to remove the tape.

Thus, during the identification techniques, electronic device 110-1 and/or computer system 120 may acquire one or more images (and, more generally, one or more measurements); identify one or more medical devices; and/or analyze the one or more medical devices (such as based at least in part on classification information, metadata and/or one or more inspection criteria). For example, electronic device 110-1 and/or computer system 120 may: identify any instances of medical device 108 in one or more images (e.g., using image analysis or deep learning); and/or analyze medical device 108 based at least in part on one or more inspection criteria (which may involve object recognition, tagging or labeling and/or counting). In some embodiments, electronic device 110-1 may display, store and/or provide a report summarizing the results of the analysis. In general, one or more of the operations associated with the identification techniques may be performed locally on electronic device 110-1 and/or remotely on computer system 120 via network 122. For example, image analysis of the one or more images may be performed remotely by computer system 120, the one or more inspection criteria may be assessed remotely by computer system 120 and/or the report summarizing the results may be stored on electronic device 110-1 and/or disseminated to recipients or one or more other electronic device or computers (such as computer system 120).

In some embodiments, electronic device 110-1 may be a portable electronic device, such as smart glasses or an augmented-reality display, and electronic device 110-1 may display the instructions or information on one or more heads-up displays associated with electronic device 110-1.

In some embodiments, one or more of the operations in the identification techniques may leverage domain understanding or knowledge associated with a different application (in the same or a different market segment). This may facilitate cross-domain understanding. For example, domain knowledge may be packaged in an ontology (e.g., represented as collection logical rules), so that it can be shared or reused by multiple applications. For example, medical devices may be described in a knowledge graph, adhering to an ontology consisting of a type hierarchy providing a nested classification; attributes describing tips, blades, handles, etc.; and/or relationships, such as subcomponents and set membership. Moreover, one or more of the operations in the identification techniques, such as business logic or the one or more inspection criteria, may be provided by a third party, which is different from the user or a provider of the identification techniques. Furthermore, domain knowledge may be aligned with images in a joint embedding space, allowing devices (such as medical devices or instruments) to be identified by comparing them to their knowledge graph descriptions.

In this way, the identification techniques may facilitate accurate and efficient identification of medical device 100. This identification may improve medical workflows. For example, the identification techniques may facilitate tray assembly with reduced errors, and thus reduced complexity or cost. Consequently, the identification techniques may improve satisfaction or the user experience of a user, such as a surgical technician.

While the preceding discussion illustrated the identification of medical device 108, in general the identification techniques may be used in or relevant to a variety of fields or market segments, including: medicine or surgery, aviation, industrial maintenance, inspection, verification, car maintenance, defense or military, remote experts, customer relationship management, retail, sales, etc. For example, the identification techniques may be used to: identify surgical or medical tools in a tray to confirm the correct number, placement, type of tools, that the tools are clean, etc.; to verify that the tools are laid out/correctly assembled; to determine which tools were used during a surgery; and/or to perform a real-time inventory (such as to count the number of tools on a tray or in a drawer).

For example, during monitoring techniques, electronic device 110-1 may execute program instructions or software that performs one or more operations. Note that the program instructions may be a standalone executable that is installed on electronic device 110-1 and executed in an environment of electronic device 110-10 (such as by an operating system on electronic device 110-1). Alternatively or additionally, program instructions may be executed in the environment of a Web browser, such as: a Web-browser plugin, a Web application, a native application leveraging one or more application programming interfaces, and/or a standalone embedded application. In some embodiments, at least a portion of the functionality associated with the monitoring techniques is implemented using a client-server architecture, e.g., by computer system 120 via WLAN 114, cellular-telephone network 116 and/or network 122). Note that the program instructions may include configuration instructions for a preinstalled augmented reality application (such as a neural network or supervised-learning model) or container on electronic device 110-1. These configuration instructions may be provided to electronic device 110-1, and may tailor or customize the preinstalled augmented reality application or container, so that, when executed, it performs the operations associated with the augmented reality application.

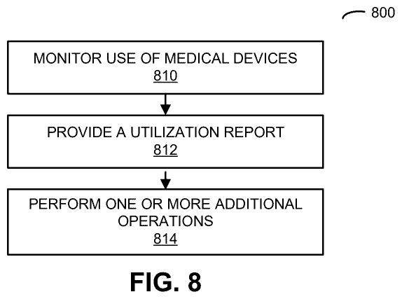

As described further below with reference to FIGS. 8-21 and 23-29, in the monitoring techniques at least one or more operations, features or capabilities in the aforementioned identification techniques may be used in monitoring techniques. Notably, an electronic device (such as electronic device 110-1) may monitor (e.g., using an image sensor, such as a CMOS sensor or a CCD sensor) use of medical devices (such as medical device 108) in a tray of medical devices or a peel pack of medical devices (which is sometimes referred to as a ‘surgical pouch,’ and which may be a sterilized package with multiple medical devices) during at least a medical procedure (such as a type of surgery). Note that the monitoring may include monitoring of a Mayo stand or a table holding at least the tray of medical devices or the peel pack of medical devices during the medical procedure. (Note that a ‘Mayo stand’ is used in surgery to prepare the set of surgical instruments or medical devices to be used in a medical procedure. These surgical instruments or medical devices are intended to be used. In general, surgical instruments or medical devices may be stored on a back table in a operating room during a medical procedure.) In some embodiments, the monitoring may involve: identifying the medical devices observed during the monitoring; and/or verifying the medical devices observed during the monitoring.

Then, electronic device 110-1 may provide, addressed to a computer system (such as computer system 120), a utilization report based at least in part on the monitoring, such as medical devices that were or were not used during the medical procedure. This utilization report may include historical usage of medical devices: in a type of the medical procedure, for a type of patient (such as one or more patients having a type of medical condition, e.g., a disease, and/or a severity of the type of medical condition) and/or by a given medical provider (such as a surgeon).

Note that the medical devices may include: a surgical instrument, a surgical screw, surgical supplies and/or a surgical machine. In some embodiments, the monitoring may include determining: a type of a medical device in the medical devices; a classification of the medical device; an identification code of the medical device (such as a SKU); membership of the medical device in a group (such as a surgical tray, peel pack or set); or a manufacturer of the medical device.

Moreover, electronic device 110-1 may document a particular or discrete moment in the medical procedure. Note that the documentation may be based at least in part by a trigger, such as: motion of a gloved hand; an absence of the gloved hand in a field of view of the image sensor; placement of a medical device in a predefined portion in a frame of the image sensor; etc. For example, a surgical technician may hold a medical device in a predefined 3D space to initiate a trigger. This trigger may result in an image of the medical device to be acquired or captured, which may function as a form of self-check out of the medical device. In general, the trigger may be explicit or implicit. Alternatively, electronic device 110-1 may perform continuous monitoring during the medical procedure. In some embodiments, electronic device 110-1 may redact information acquired during the monitoring, such as protected health information associated with a patient or an identity of the medical provider. (Alternatively, the redaction of the information may be performed by computer system 120 based at least in part in information provided by electronic device 110-1.)

Furthermore, during the monitoring, electronic device 110-1 may provide an instruction for an individual (such as a nurse, a surgical technician, a surgeon, etc.) to change a perspective (such as an orientation or a field of view) of the image sensor during the medical procedure. This instruction may ensure that area(s) of interest (such as an edge of a towel, a table holding at least the tray of medical devices or the peel pack of medical devices, etc.) is included in a field of view of the image sensor during the monitoring. Alternatively or additionally, during the monitoring, electronic device 110-1 may automatically change the field of view to dynamically track the position of area(s) of interest during the medical procedure.

Additionally, based at least in part on the historical usage of medical devices: in the type of the medical procedure, for the type of patient and/or by the given medical provider, electronic device 110-1 (and/or computer system 120) may provide a recommendation for one or more medical devices to be included in (or removed from) another instance of the tray of medical devices or the peel pack of medical devices for use in a future medical procedure. For example, a list of medical devices and surgical instruments specified in a case-cart, count sheet and/or preference card may be updated based at least in part on the recommendation. As described further below, information in the utilization report and/or this recommendation may be used in the assembly techniques. Note that, in some embodiments, the recommendation may be based at least in part on a preference of the given medical provider, such as preference of a surgeon for a particular surgical instrument during a particular type of surgery and/or based at least in part on a predefined surgical plan (which may be generated in advance of the type of surgery). In some embodiments, the recommendation is provided to computer system 120, which may implement EMR software. As described further below, in some embodiments this EMR software may use the recommendation in the assembly techniques.

In some embodiments, during at least the medical procedure, electronic device 110-1 may predict a medical device will be needed in the medical procedure, where the medical device is not included in the tray of medical devices or the peel pack of medical devices. Then, electronic device 110-1 may provide an instruction to obtain the medical device or may automatically obtain the medical device. For example, the instruction may be provided to a vending machine with medical devices in real-time during the medical procedure.

Moreover, during the assembly techniques, electronic device 110-1 may execute program instructions or software that performs one or more operations. Note that the program instructions may be a standalone executable that is installed on electronic device 110-1 and executed in an environment of electronic device 110-10 (such as by an operating system on electronic device 110-1). Alternatively or additionally, program instructions may be executed in the environment of a Web browser, such as: a Web-browser plugin, a Web application, a native application leveraging one or more application programming interfaces, and/or a standalone embedded application. In some embodiments, at least a portion of the functionality associated with the monitoring techniques is implemented using a client-server architecture, e.g., by computer system 120 via WLAN 114, cellular-telephone network 116 and/or network 122). Note that the program instructions may include configuration instructions for a preinstalled augmented reality application (such as a neural network or supervised-learning model) or container on electronic device 110-1. These configuration instructions may be provided to electronic device 110-1, and may tailor or customize the preinstalled augmented reality application or container, so that, when executed, it performs the operations associated with the augmented reality application.

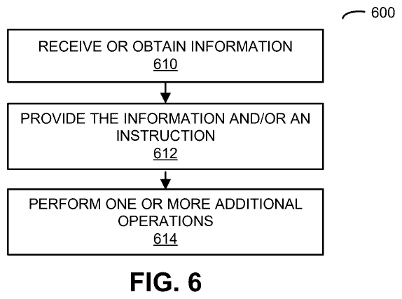

Notably, as described further below with reference to FIGS. 6-7 and 22, in the assembly techniques knowledge about usage (or not) of medical devices (such as a surgical instrument, a surgical screw, surgical supplies or a surgical machine), e.g., during one or more surgeries and, more generally, in one or more medical procedures, may be used in assembly techniques. Notably, an electronic device (such as electronic device 110-10 may receive or obtain, from a computer system (such as computer system 120), information specifying medical devices to include in the tray of medical devices or the peel pack of medical devices based at least in part on historical usage of medical devices: in a type of medical procedure (such as a type of surgery), for a type of patient (such as one or more patients having a type of medical condition, e.g., a disease, and/or a severity of the type of medical condition) and/or by a given medical provider (such as a surgeon). Then, electronic device 110-1 may provide (e.g., on a display in or associated with electronic device 110-1), the information and/or an instruction to assemble the tray of medical devices or the peel pack of medical devices based at least in part on the information. For example, a number of the medical devices included in the information for use in the tray of medical devices or the peel pack of medical devices may be reduced relative to a second number of medical devices in a predefined set of medical devices.

In this way, the assembly techniques may facilitate intelligent tray or peel-pack assembly. This may reduce the cost of assembly and/or the size or complexity of the tray of peel pack, which in turn may reduce delays and/or errors in assembly and/or use of medical devices during the medical procedure. For example, the assembly techniques may enable dynamic, local (i.e., hospital-based) or industry-level optimization of tray or peel pack assembly.

Note that the information may include: a type of a medical device in the medical devices; a classification of the medical device; an identification code of the medical device (such as a SKU); membership of the medical device in a group (such as a surgical tray, peel pack or set); or a manufacturer of the medical device. For example, the information may include: a barcode of one of the medical devices; a SKU of a second one of the medical devices; and/or visual attributes of a third one of the medical devices. Moreover, the information may include an RF identifier of a fourth medical device in the medical devices. Electronic device 110-1 may identify the fourth medical device based at least in part on the RF identifier. In some embodiments, the information for a given medical device in the medical devices may be associated with multiple locations on the given medical device.

Furthermore, in some embodiments, electronic device 110-1 )or an associated robot) may automatically assemble the tray of medical devices or the peel pack of medical devices based at least in part on the information. For example, automatically assembling the tray of medical devices or the peel pack of medical devices may include: identifying a medical device based at least in part on the inclusion of the medical device in the information; selecting the medical device; and placing the medical device at a given location and/or orientation on the tray of medical devices or in the peel pack of medical devices. Note that electronic device 110-1 may include an image sensor (such as a CMOS sensor or a CCD sensor) that acquires one or more images, and electronic device 110-1 may identify the medical device based at least in part on the one or more images (such as by using a pretrained machine-learning model, e.g., a pretrained neural network). Moreover, the medical device may be identified based at least in part on a visual attribute of the medical device, such as: a shape of at least a portion of the medical device, a texture of at least a second portion of the medical device; and/or a reflectivity of at least a third portion of the medical device. However, the disclosed assembly techniques are not limited to descriptors such as shape, texture and/or reflectivity. More generally, the assembly techniques may use embedding of multiple descriptors, such as 512 descriptors.

The identifying may be performed by electronic device 110-1. Alternatively or additionally, electronic device 110-1 may: provide the one or more images to computer system 120; and receive the identification of the medical device from computer system 120.

Note that, prior to the identifying, electronic device 110-1 may: scale a size of an image in the one or more images; and rotate the scaled image to maximize second information associated with pixels in a barcode or text associated with the medical device. When the information includes a barcode, electronic device 110-1 may assess quality of a first image in the one or more images and, based at least in part on the assessed quality, may selectively adjust a zoom of the image sensor.

We now describe embodiments of a method in the identification techniques. FIG. 2 presents a flow diagram illustrating an example of a method 200 for identifying a medical device using an electronic device, such as electronic device 110-1 in FIG. 1.

During operation, the electronic device may receive or obtain a first image of the medical device (operation 210), where at least a portion of the medical device is partly obscured by an object in the first image or is damaged or degraded. Then, the electronic device may identify the medical device (operation 212) based at least in part on information associated with a second portion of the medical device that is not partially obscured by the object or that is not damaged or degraded, where the information includes a visual attribute of the medical device.

Note that the information may include: a barcode of the medical device, a SKU of the medical device, a shape of at least the second portion of the medical device, a texture of the medical device; and/or a reflectivity of the medical device. Note that visual attributes, such as shape, texture, etc., may be latent features in the embedding space. Latent visual attributes in embedding space may or may not be explicitly projected into categorical variables or natural language. Moreover, the medical device may include a surgical instrument, a surgical screw, surgical supplies and/or a surgical machine. Furthermore, the information may include: a subset of text on a first surface (such as a front or a back surface) of the medical device; and/or a subset of a barcode on a second surface of the medical device (such as a front or back surface). Additionally, the damage or degradation may include a scratch. In some embodiments, the identification may include: a type of the medical device; membership of the medical device in a surgical tray or set; a classification of the medical device; an identification code of the medical device (such as a SKU); and/or a manufacturer of the medical device.

In some embodiments, the electronic device optionally performs one or more additional operations (operation 214). For example, identifying the medical device (operation 212) may include determining the second portion. Moreover, identifying the medical device (operation 212) may include masking the first image based at least in part on the determined second portion, where the masked first image includes the determined information, and the medical device is identified based at least in part on the masked first image.

In some embodiments, identifying the medical device (operation 212) may include: providing the first image to the computer; and receiving, from the computer, second information identifying the medical device.

Moreover, the electronic device may determine an RF identifier of the medical device. The electronic device may identify the medical device (operation 212) based at least in part on the RF identifier. For example, an electronic device may detect a set of medical devices when the RF identifier is in range. In order to uniquely identify the medical device from the set, the remedial action may be to prompt the user to circle or point and then to select the most-similar instrument or medical from the set based at least in part on the available information.

Furthermore, the object may include a portion of a human hand or another medical device in the first image. However, in some embodiments, the obstruction may be associated with an incorrect camera angle or perspective when the first image was acquired. Based at least in part on the portion of the medical device, the electronic device may, prior to identifying the medical device (operation 212), selectively perform a remedial action to facilitate identification of the medical device. For example, the first image may be rejected when the object partially obscures the information. When the first image is rejected, the electronic device may provide an instruction to acquire a second image of the medical device using the image sensor without the partial obstruction of the information before the medical device is identified. Notably, the instruction for acquiring the second image may be to acquire the second image without the obstruction and/or to acquire the second image with additional information that facilitates the identification (such as of the other side of the medical device, with a different perspective or angles to the medical device, etc.). Thus, the instruction for the second image may include flipping the medical device, opening a pair of scissors, focusing on a tip of the medical device, etc. Note that identification from visual attributes may result in two candidate medical devices differing only in size. When this occurs, the instruction for the second image may include the remedial action of adding a reference object to the scene, such as a ruler or fiducial marker (e.g. an Open Source Computer Vision Library (OpenCV) ArUco marker), to establish scale, so that the size of the medical device may be recovered and compared against the known dimensions of the medical device. Alternatively or additionally, the remedial action may include an instruction to point to the information or providing a circle of identification or color coding of the first image to guide acquisition of the second image of the medical device. The remedial action may be to request that the user clean the medical device or remove tape from the device before acquiring the second image. In some embodiments, the remedial action may be to create the second image by redacting a portion of the first image. In general, adding a scale or a reference may be used in one or more other embodiments to assist in reducing the number of possible candidate medical devices or instruments during identification.

Additionally, the electronic device may provide an instruction for assembly of at least a portion of a tray, a peel pack, a case cart, a shelf, etc. based at least in part on the identified medical device.

Note that, prior to the identifying (operation 212), the electronic device may: determine a set of possible medical devices that are consistent with the information; provide (e.g., to another electronic device or a computer) third information specifying the set of possible medical devices; and receive feedback (e.g., from the other electronic device or computer) about one or more of the set of possible medical devices, where the identifying (operation 212) is based at least in part on the feedback. Note that the third information specifying a set of possible medical devices may come from one or more other sources, such as: devices within a class or category, devices in a certain range of sizes, devices belonging to a certain manufacturer, devices on the network, and/or devices belonging to a tray or set.

In some embodiments, the information may include a subset of a barcode or text. Prior to the identifying (operation 212), the electronic device may: scale a size of the image; crop the image to contain the text or code and rectify the crop with a transformation, such as a homography; rotate the scaled image to maximize fourth information associated with pixels in the barcode or text; and/or to match against a template.

Note that the identifying (operation 212) may include estimating the portion of the medical device based at least in part on the second portion of the medical device, where the identifying is based at least in part on the estimated portion of the medical device.

Moreover, the electronic device may recognize the information at multiple locations on or associated with the medical device.

When the information includes a marking, such as a barcode or text, the electronic device may assess quality of the first image and, based at least in part on the assessed quality, selectively: adjusting an optical zoom of the image sensor to acquire more pixels; modulating a light source to eliminate glare; applying focus stacking to increase the depth of field; registering multiple images from different camera poses, and with different exposure settings into a single image to increase information; etc. This may all be done while acquiring an image on every frame and repeatedly attempting to decode the marking at a rate of up to 30 times per second. Furthermore, the user may be instructed to tilt the instrument to find an angle of incidence for which the etching reflects light, hold the camera still to allow multi-image bursts; slowly change the camera pose, etc.

Furthermore, the electronic device may reduce a false positive rate in the identification (operation 212) by performing the identification based at least in part on the image and the information, where the information may include a partial identifier of the medical device.

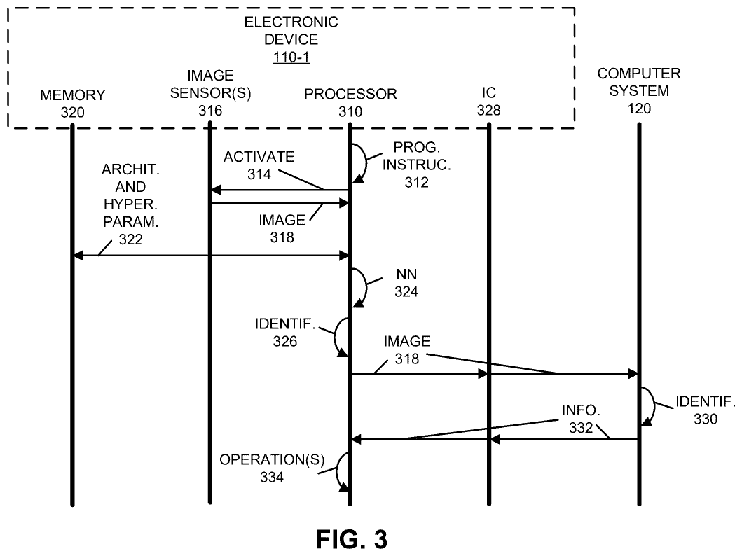

FIG. 3 presents a drawing illustrating an example of communication among electronic device 110-1 and computer system 120. During operation, processor 310 in electronic device 110-1 may execute program instructions 312. In response, processor 310 may activate 314 one or more image sensors 316 in electronic device 110-1 and may receive an image 318 of a current field of view (FOV) of a medical device in at least one of the one or more image sensors 316.

Then, processor 310 may identify 326 the medical device based at least in part on analysis of the one or more images 318. The identification 326 may be performed by processor 310. For example, processor 310 may implement a neural network (NN) 324 based at least in part in a configuration of the neural network 324 (such as based at least in part on an architecture and hyperparameters 322 stored in memory 320 in electronic device 110-1) that identifies 326 the medical device. (Note that neural network 324 may be pretrained and/or fine-tuned. A pretrained neural network is a model that has been trained on a large amount of data on a general task. Device identification typically calls for a ‘fine-tuned’ neural network, which is the result of additional training of a pretrained model on a domain specific dataset, such as medical devices, to learn a specific task, such as identification. In general, a machine-learning model must be trained before it is used, hence it is understood that a neural network is trained before it is used in a system for performing inference. In some embodiments, the training and/or fine-tuning may include transfer learning and/or reinforcement learning.) Alternatively or additionally, processor 310 may provide the one or more images 318 to computer system 120 using interface circuit (IC) 328 in electronic device 110-1. In response, computer system 120 may perform identification 330 and may provide information 332 that specifies at least the medical device to interface circuit 322, which then provides information 332 to processor 310.

In some embodiments, processor 310 may perform one or more additional operations 334. For example, processor 310 may display information 326 on a display (not shown) in electronic device 110-1.

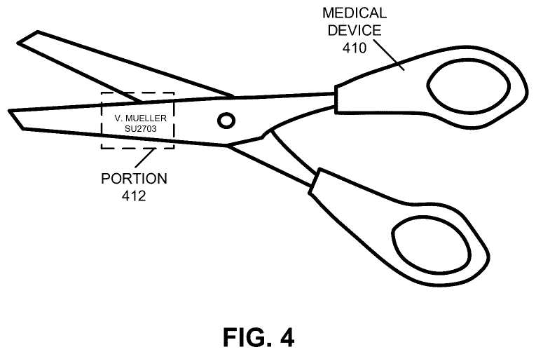

We now further describe embodiments of the identification techniques. FIG. 4 presents a drawing illustrating an example of a medical device 410, such as surgical instrument (e.g., a pair of scissors). This medical device includes a portion 412 with a SKU and manufacturer information.

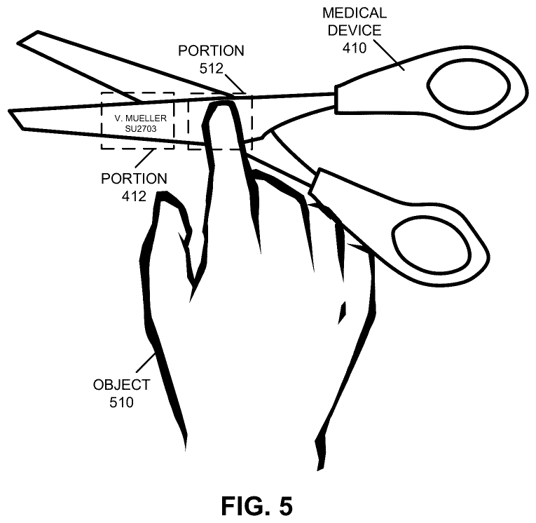

Alternatively, as shown in FIG. 5, which presents a drawing illustrating an example of medical device 410, an object 510 (such as a human hand or finger) may obscure a portion 512 of medical device 410. However, using the identification techniques, portion 412 may be determined and information (such as the SKU and the manufacturer information) may be used to identify medical device 410.

In some embodiments, the identification techniques may be used to mask or crop an image of medical device 410 to isolate portion 412 with the information. Note that the identification techniques may be used to redact a portion of the medical device, e.g., by ‘masking out’ (rather than ‘masking in’) a particular region with damage, an obscuring substance, or confounding information. Moreover, in some embodiments, multi-modal identification may be performed. For example, medical device 410 may be identified based at least in part on: at least a portion of a barcode, a SKU, at least a portion of a shape of medical device 410, and/or an RF identifier of medical device 410.

The identity of medical device 410 may be used in different workflows. For example, the identity may be used to facilitate easier tray assembly, or to facilitate the usage tracking of medical devices in the operating room during surgery. Alternatively or additionally, the identity may be provided to a medical system, which may use this information in another workflow.

In some embodiments, the identification techniques may be implemented using a general-purpose camera plug-in module or a camera software module (or set of instructions) for a portable electronic device, which may be shared by different mobile applications supporting different workflows. Moreover, in some embodiments, the identification techniques may be implemented using a software development kit (SDK) or an application programming interface (API).

In some embodiments, the identification techniques may leverage an object detector to recognize surgical instruments, various barcodes, text, and human hands. For example, the identification techniques may use the Yolo object detection technique for real-time identification and localization of objects within an image or video stream. Yolo employs a single-stage, fully CNN architecture. This may enable rapid object identification and bounding box creation in a single forward pass, achieving significantly faster processing speeds compared to other approaches. The efficiency of Yolo may stem from its unique approach to object detection. Instead of treating the task as a classification problem, Yolo frames it as a regression problem. The CNN may simultaneously predict bounding box coordinates and class probabilities for each object in an image. This unified architecture may eliminate the need for separate classification and localization stages, leading to drastically reduced processing times. The real-time capabilities of Yolo may make it suitable for real-time on-device applications.

The disclosed identification techniques may group detections such that barcodes and text are only processed when they lie on a medical device and are not fully or partially occluded (e.g., by hands). This may allow an electronic device to know which medical devices were identified and to avoid spurious text or codes in the scene that do not relate to surgical instruments. This may also enable the medical device to identify multiple medical devices simultaneously. Note that human hands are a common source of occlusion. Tape and printed labels are another source of occlusion. An object detector may recognize occlusions in the scene and may check that the text, barcode, or surgical instrument (with respect to the modality or modalities used for identification) are not occluded.

In some embodiments, an image may be rejected when a hand (or an obscuring object) is in the way and/or based at least in part on a number of pixels used to capture the image. Alternatively, available information that is visible may be used, while the occluding object may be masked out, e.g., with black pixels to avoid confounding. Moreover, the identification techniques may automatically adjust: the focus, exposure, frame rate, white balance, and/or zoom to better capture interesting features or elements of a given medical device. Note that user feedback may be used in decoding. Furthermore, in the identification techniques, a check may be performed to confirm that an identified surgical instrument and the SKU in the image match.

Additionally, in some embodiments, potential matches may be provided. Notably, the problem being addressed is when visual recognition fails because the relevant portion(s) of an image is obscured, damaged, or degraded. Examples include recognizing an object based at least in part on text, barcode, shape, and/or appearance. The solution may use other discernible visual attributes that can narrow down the set of possible matches and then prompt a user to enrich information (or to provide feedback), so that the electronic device can obtain a unique answer.

For example, a medical device may be identified from an image of a partially obscured or degraded square Data Matrix barcode. Assume the database or data structure has a catalog of medical devices with corresponding Global Trade Item Number (GTIN) codes. Furthermore, assume the database or data structure has pictures and descriptions of the medical devices to aid in identification.

For each medical device in the catalog, generate a Data Matrix image based on the corresponding GTIN code. Note that a Data Matrix may include a grid of white and black cells with rows and columns ranging from 10×10 to 144×144.

Then, the electronic device may register the partially obscured barcode to the pixel grid. For best results, this may require a minimum 9 pixels per cell. Thus, a 16×16 grid may require a 48×48 pixel image or larger. For example, find two corners of the barcode in the image and two straight lines. This may involve running an edge or a corner detector, and performing image morphology (e.g., dilation) on the image, etc. Next, constraints may be used to solve for the projection matrix to map pixels into the grid.

For each cell (x, y) in the grid, classify the cell as (x, y) as 1 or 0 using a function of pixel intensity, e.g., threshold on average intensity. Some of these grid values may be in obscured or degraded regions, but other regions may be usable.

Now, we have a grid G1 computed when a given cell was classified as 1 or 0 using the function of pixel intensity and a set of grids {G} computed when the medical device was identified from the image of the partially obscured or degraded square Data Matrix barcode. We wish to search {G} for the ‘closest; match to G1. For example, we could ‘tokenize; each grid using a sliding window of fixed k×k subgrids and then perform a bag-of-words match based at least in part on Jaccard Similarity to obtain the top-N candidates. Each possible subgrid may be mapped to an integer between I={1, . . . k·k} and a grid may be represented by a bag G={x₁, . . . xₙ} where xᵢ is in I. Note that a technique such as MinHash or the min-wise independent permutations locality sensitive hashing technique can make the catalog search based at least in part on Jaccard Similarity more feasible.

Given N candidate matches, the electronic device can re-rank the candidates to find the best M less than N candidates (where M and N are non-zero integers) by checking each candidate pairwise. For example, we could look for the largest matching contiguous region.

Moreover, for each of the M candidates, the user may select the best candidate based at least in part on the pictures (or images) and descriptions from the catalog.

In another example, a medical device may be identified based at least in part on text. Notably, given the scenario in the preceding example, assume we are matching medical devices against a catalog based at least in part on text on the medical device (such as a catalog number representing a sequence of ASCII characters). Let us call this a catalog identification string.

Optical character recognition may be performed to obtain rows of sequential characters. There may be gaps because of the occlusion or degradation of the text marked onto the medical device.

Moreover, a similar search may be performed using n-grams instead of windows to find the catalog identification string most similar to a row of text from the medical device.

Then, perform a re-rank based at least in part on a common substring.

Furthermore, for each of the M candidates, the user may select the best candidate based at least in part on the pictures (or images) and descriptions from the catalog.

In another example, a medical device may be identified based at least in part on appearance or shape. Notably, given the scenario in the preceding examples, assume we are matching medical devices against a catalog based at least in part on photographs or images of a portion or the whole medical device. A recognition technique that uses visual attributes (such as shape, texture, material properties, etc.) may be used to match a medical device to a reference dataset, database or data structure when the object is only partially visible.

Unlike other examples, because we are not reading a code in this example, we are not concerned with degradation of markings on the medical device. The main concern may be occlusion, especially, if the occlusion is from a different medical device. Occlusion could also result from a human hand or an obscuring substance, such as tape, on the medical device. For example, a forceps may rest on top of a needle holder. The presence of two surgical instruments may confound the identification.

In order to address this problem, the electronic device may detect occlusion. For example, an object detector may find objects of different classes that overlap. Similarly, the electronic device may employ a depth sensor, such as LiDAR, to detect occlusion. Note that, when the medical device is known to be in a z range, any pixel occupied closer than z may belong to an occluding object.

In the case of near or overlapping medical devices, the electronic device may prompt the user as to which one to identify by selecting a point on the medical device. The electronic device then uses a segmentation technique (such as an instance object detector or depth-based LiDAR) to define the boundary of both medical devices and the medical device the user does not wish to identify.

For general occlusion (or blocking or closing something), which may include obscuring (or preventing something from being seen), there may be one or more mitigation options. For example, visual cues may be provided for the user to move the occluding object, capturing the image only when occlusion is resolved. Alternatively, the occluding object may be masked using black pixels to reduce potential confounding. In some embodiments, ‘self-occlusion’ may be detected based at least in part on a shape of the object or medical device (e.g., we may obtain get a mask of the object from an object segmentation model). Using this information, the electronic device can detect that an object is on its side and not fully visible. Then, the user may be notified to rotate the medical device, open the medical device so that blades are exposed, etc., to get a better view for identification.

Moreover, occlusion may be avoided by asking the user to place the medical device on a physical mat of known size and color. The mat may have markings showing where the medical device should be placed. A mat may have fiducial markings to improve the accuracy of estimating the transform from pixel to known dimensions on the mat. An image may be acquired automatically when: a single medical device is detected on the mat; no other objects are detected on the mat; and/or when the human hand no longer obscures the mat. The known size and color of the mat may allow the electronic device to recover scale and hue (because the mat color may serve as a reference to recover white for white balance), thereby narrowing the set of possible instruments by size and color. The guides on how to place the medical device may mitigate self-occlusion. The electronic device may acquire an image whenever the hand leaves the mat, ensuring hand-occlusion is avoided. The requirement that an image is only acquired when a single instrument is on the mat also prevents occlusion from another medical device.

We now describe the monitoring and/or assembly techniques. Note that the monitoring and/or assembly techniques may include or may use at least a portion of the operations and capabilities disclosed in the embodiments of the identification techniques.

As discussed previously, it can be difficult to accurately and efficiently assemble a tray of medical devices or a peel pack of medical devices. This complexity can result in waste, increased assembly time, increased cost, and errors, both during the assembly process and during a subsequent medical procedure.

As described below with reference to FIGS. 6-29, in order to address these problems, an electronic device (such as electronic device 110-1) may, at least in part, perform the monitoring and/or assembly techniques. Notably, FIG. 6 presents a flow diagram illustrating an example of a method 600 for facilitating assembly of a tray of medical devices or a peel pack of medical devices using an electronic device, such as electronic device 110-1 in FIG. 1.

During operation, the electronic device may receive or obtain, from a computer system, information (operation 610) specifying medical devices to include in the tray of medical devices or the peel pack of medical devices based at least in part on historical usage of medical devices: in a type of medical procedure (such as a type of surgery), for a type of patient (such as one or more patients having a type of medical condition, e.g., a disease, and/or a severity of the type of medical condition) and/or by a given medical provider (such as a surgeon). Then, the electronic device may provide (e.g., on a display associated with the electronic device), the information and/or an instruction (operation 612) to assemble the tray of medical devices or the peel pack of medical devices based at least in part on the information.

Thus, a number of the medical devices included in the information for use in the tray of medical devices or the peel pack of medical devices may be reduced relative to a second number of medical devices in a predefined set of medical devices.

Note that the medical devices may include: a surgical instrument, a surgical screw, surgical supplies and/or a surgical machine. In some embodiments, the information may include: a type of a medical device in the medical devices; a classification of the medical device; an identification code of the medical device (such as a SKU); membership of the medical device in a group (such as a surgical tray, peel pack or set); or a manufacturer of the medical device.

Moreover, the information may include: a barcode of a first medical device in the medical devices; a SKU of a second medical device in the medical devices; and/or visual attributes of a third medical device in the medical devices. Furthermore, the information may include an RF identifier of a fourth medical device in the medical devices. The electronic device may identify the fourth medical device based at least in part on the RF identifier. Additionally, the information for a given medical device in the medical devices may be associated with multiple locations on the given medical device.

In some embodiments, the electronic device optionally performs one or more additional operations (operation 614). For example, the electronic device (or an associated robot) may automatically assemble the tray of medical devices or the peel pack of medical devices based at least in part on the information. Note that automatically assembling the tray of medical devices or the peel pack of medical devices may include: identifying a medical device based at least in part on the inclusion of the medical device in the information; selecting the medical device; and placing the medical device at a given location and orientation on the tray of medical devices or in the peel pack of medical devices. The electronic device may include an image sensor that acquires one or more images, and the electronic device may identify the medical device based at least in part on the one or more images. Moreover, the medical device may be identified based at least in part on a visual attribute of the medical device, such as: a shape of at least a portion of the medical device, a texture of at least a second portion of the medical device; and/or a reflectivity of at least a third portion of the medical device. However, the disclosed assembly techniques are not limited to descriptors such as shape, texture and/or reflectivity. More generally, the assembly techniques may use embedding of multiple descriptors, such as 512 descriptors.

The identifying may be performed by the electronic device. Alternatively or additionally, the electronic device may: provide the one or more images to the computer system; and receive the identification of the medical device from the computer system.

Similarly, in some embodiments, instead of receiving or obtaining the information from the computer system, the electronic device may receive or obtain the information from memory in or associated with the electronic device.

Furthermore, prior to the identifying, the electronic device may: scale a size of an image in the one or more images; and rotate the scaled image to maximize second information associated with pixels in a barcode or text associated with the medical device. When the information includes a barcode, the electronic device may assess quality of a first image in the one or more images and, based at least in part on the assessed quality, may selectively adjust a zoom of the image sensor.

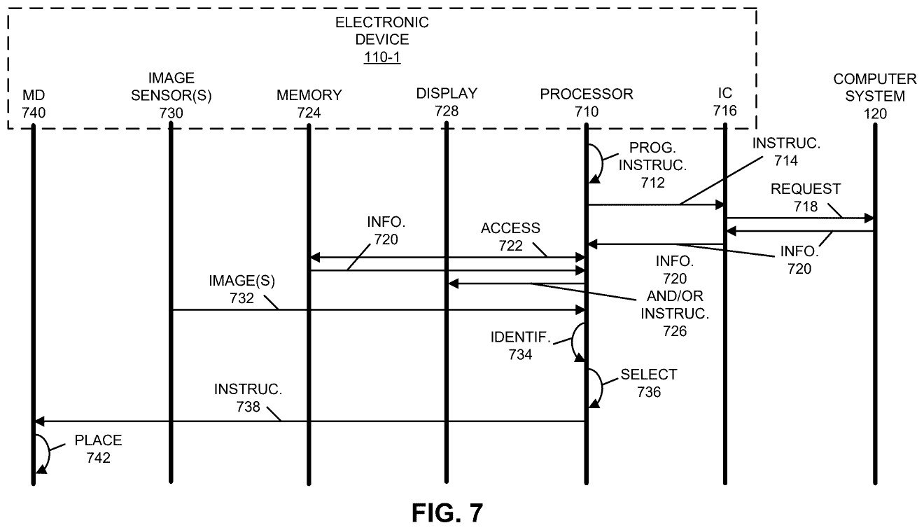

FIG. 7 presents a drawing illustrating an example of communication between electronic device 110-1 and computer system 120. During operation, a processor 710 in electronic device 110-1 may execute program instructions 712. In response, processor 710 may instruct 714 an interface circuit 716 in electronic device 110-1 to request 718 information 720 from computer system 120. Then, interface circuit 716 may receive information 720 from computer system 120. Alternatively, processor 710 may obtain information 720 by accessing 722 information 720 in memory 724 in electronic device 110-1. Note that information 720 may specify medical devices to include in the tray of medical devices or the peel pack of medical devices based at least in part on historical usage of medical devices: in a type of medical procedure, for a type of patient; by a given medical provider; and/or inventory (such as an inventory of medical devices at a hospital).

Next, processor 710 may provide information 718 and/or an instruction 726 to assemble the tray of medical devices or the peel pack of medical devices based at least in part on information 718. For example, processor may display information 718 and/or instruction 722 on a display 728 in or associated with electronic device 110-1. A surgical technician or a nurse may assemble the tray of medical devices or the peel pack of medical devices based at least in part on information 718 and/or instruction 726.

Alternatively or additionally, providing information 718 and/or instruction 726 may be provided to one or more components in electronic device 110-1 (or an associated robot that communicates with electronic device 110-1) to automatically assemble the tray of medical devices or the peel pack of medical devices based at least in part on information 718 and/or instruction 738. For example, one or more image sensors 730 in electronic device may acquire one or more images 732 of a medical device and processor 710 may identify 734 the medical device based at least in part on the one or more images 732 and inclusion of the medical device in information 718. For example, processor 710 may identify 734 the medical device using the one or more images 732 and a pretrained machine-learning model, such as a pretrained neural network. Then, processor 710 may select 736 the medical device and may provide an instruction 738 to a mechanical device (MD) 740 (such as a mechanical gripper) in or associated with electronic device 110-1. Next, based at least in part on instruction 738, mechanical device 740 may place 742 the medical device at a given location and orientation on the tray of medical devices or in the peel pack of medical devices.

FIG. 8 presents a flow diagram illustrating an example of a method 800 for monitoring usage of medical devices during a medical procedure using an electronic device, such as electronic device 110-1 in FIG. 1. During operation, the electronic device may monitor use of medical devices (operation 810) in a tray of medical devices or a peel pack of medical devices during at least a medical procedure. Then, the electronic device may provide, addressed to a computer system, a utilization report (operation 812) based at least in part on the monitoring, where the utilization report includes historical usage of medical devices: in a type of the medical procedure, for a type of patient and/or by a given medical provider (such as a surgeon).

Note that the medical devices may include: a surgical instrument, a surgical screw, surgical supplies and/or a surgical machine. In some embodiments, the monitoring (operation 810) may include determining: a type of a medical device in the medical devices; a classification of the medical device; an identification code of the medical device (such as a SKU); membership of the medical device in a group (such as a surgical tray, peel pack or set); or a manufacturer of the medical device. Moreover, the medical procedure may include a type of surgery. Furthermore, the type of patient may have a type of medical condition (such as a disease) and/or a severity of the type of medical condition.

Furthermore, the monitoring (operation 810) may include monitoring of a Mayo stand or a table holding at least the tray of medical devices or the peel pack of medical devices during the medical procedure.

Note that the electronic device may document a particular or discrete moment in the medical procedure. The documentation may be based at least in part by a trigger, such as: motion of a gloved hand; an absence of the gloved hand in a field of view of the image sensor; placement of a medical device in a predefined portion in a frame of the image sensor; etc. In general, the trigger may be explicit or implicit. Alternatively, the electronic device may perform continuous monitoring during the medical procedure.

In some embodiments, the electronic device optionally performs one or more operations (operation 814). For example, the electronic device may provide a recommendation for one or more medical devices to be included in another instance of the tray of medical devices or the peel pack of medical devices for use in a future medical procedure. This recommendation may be based at least in part on the historical usage of medical devices: in the type of the medical procedure, for the type of patient and/or by the given medical provider. Note that the recommendation may be based at least in part on a preference of the given medical provider. In some embodiments, the utilization report and/or the recommendation may be provided to the computer system, which may implement EMR software. The EMR software may store and/or subsequently use the utilization report and/or the recommendation.

Additionally, the electronic device may predict a medical device will be needed in the medical procedure, where the medical device is not included in the tray of medical devices or the peel pack of medical devices. Then, the electronic device may provide an instruction to obtain the medical device or may automatically obtain the medical device.

Note that the electronic device may redact information acquired during the monitoring (operation 810), such as protected health information associated with a patient or an identity of the medical provider. For example, the redacted information may be obscured or covered with black pixels.

Moreover, during the monitoring (operation 810), the electronic device may provide an instruction to change a perspective (such as an orientation or a field of view) of an image sensor (e.g., in or associated with the electronic device) during the medical procedure. This instruction may ensure that areas of interest (such as an edge of a towel, a table holding at least the tray of medical devices or the peel pack of medical devices, etc.) is included in a field of view of the image sensor during the monitoring (operation 810).

Furthermore, the monitoring (operation 810) may involve: identifying the medical devices observed during the monitoring (operation 810); and/or verifying the medical devices observed during the monitoring (operation 810).

In some embodiments of method 200 (FIG. 2), method 600 (FIG. 6) and method 800, there may be additional or fewer operations. For example, the electronic device may determine or detect that the medical device is present in the image prior to performing the identification (operation 212). Furthermore, the order of the operations may be changed, and/or two or more operations may be combined into a single operation. While some of the preceding embodiments may involve feedback or information received from a user, in other embodiments one or more of these operations may be automated, i.e., performed without human action.

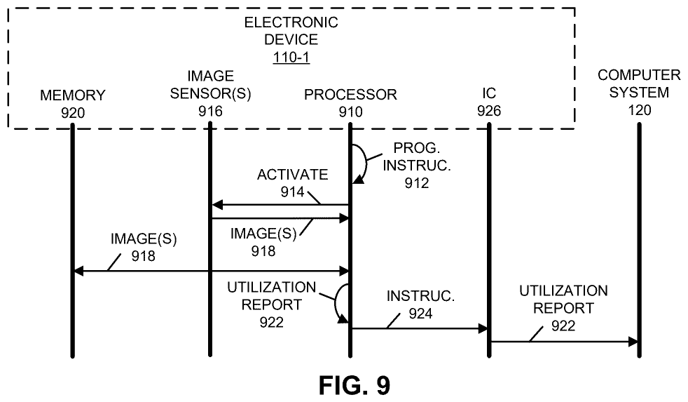

FIG. 9 presents a drawing illustrating an example of communication between electronic device 110-1 and computer system 120. During operation, processor 910 in electronic device 110-1 may execute program instructions 912. In response, processor 910 may activate 914 one or more image sensors 916 in electronic device 110-1 and may receive one or more images 918 of a current field of view (FOV) of at least one of the one or more image sensors 916 of use of medical devices or a peel pack of medical devices during at least a medical procedure. Note that the one or more images 918 may be captured or acquired based at least in part on a trigger, such as: motion of a gloved hand; an absence of the gloved hand in a field of view of at least one of the one or more image sensors 916; placement of a medical device in a predefined portion in a frame of at least one of the one or more image sensors 916; etc. (in general, the trigger may be explicit or implicit). Alternatively, the one or more images 918 may be captured or acquired continuously.

Then, processor 910 may optionally store the one or more images 918 in memory 920 in or associated with electronic device 110-1.

Next, processor 910 may instruct 924 an interface circuit 926 in electronic device 110-1 to provide, addressed to computer system 120, a utilization report 922 based at least in part on the one or more images 918. Note that utilization report 922 may include historical usage of medical devices: in a type of the medical procedure, for a type of patient and/or by a given medical provider. In some embodiments, processor 910 may generate utilization report 922 by access stored information (such as the one or more images 918) in memory 920.

While FIGS. 3, 7 and 9 illustrate communication between components using unidirectional or bidirectional communication with lines having single arrows or double arrows, in general the communication in a given operation in this figure may involve unidirectional or bidirectional communication. Moreover, while at least some of the operations in FIGS. 3, 7 and 9 are illustrated as being performed sequentially, in other embodiments two or more of the operations in FIG. 3, 7 or 9 may be performed, at least in part, in parallel.

We now further describe the monitoring and assembly techniques. The monitoring techniques may allow the tracking and analysis of activities and events, and may supply usage in, e.g., operating rooms on a large scale. In the assembly techniques, this usage information may allow medical devices to be assembled into trays and/or peel packs in an intelligence and efficient manner.

Note that approximately 70% of items (such as medical devices) prepared for surgeries are either unused and discarded or need to be reprocessed before they can be used in a subsequent surgery. Thus, there is an opportunity to provide higher quality surgical trays and/or peel packs, with: fewer delays; shorter turnover; reduced waste; and to optimize use of labor (such as nurses and surgical technicians).

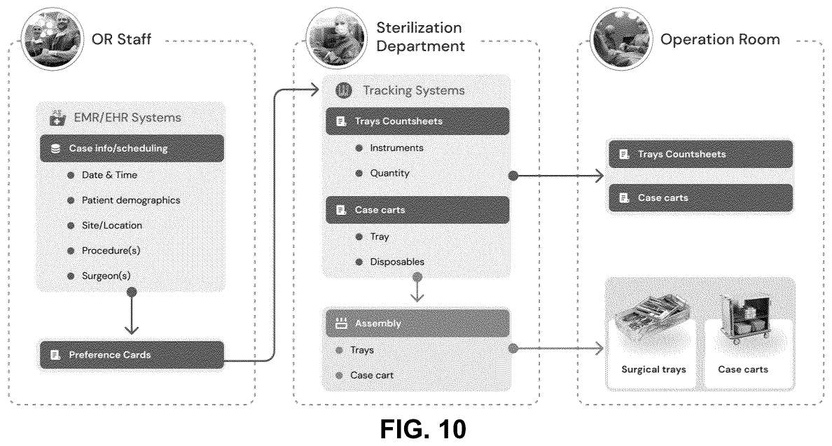

FIG. 10 presents a drawing illustrating an example of a flow associated with a surgical procedure. This flow may be performed prior to a surgery by operating-room staff (who may use EMR software or an electronic health record or EHR system) to generate instructions of medical devices to include in a tray or a peel pack, a sterilization department (which assembles trays, peel packs or case carts based at least in part on the instructions, information in tracking system, a preference card of trays of medical devices, etc.) and in an operation room (e.g., by a nurse or a surgical technician, which receive trays and case carts, and associated count sheets of medical devices). Note that the support staff (such as a surgical technician) may try to ensure that a given tray matches the spirit of a count sheet and, thus, may include medical devices that are functionally equivalent to those in a count sheet. Moreover, a given preference card may include static information and, thus, may not be accurate. Furthermore, note that a case cart may include multiple trays and disposable items (such as gowns or gloves), which is typically inventoried against a static bill of materials. Each of the trays may have an associated count sheet. However, there can be variance between instances of a count sheet for a given tray, or uncertainty in a given count sheet. In general, a ‘count sheet’ may include an ordered list of medical devices in which like items (or medical devices) are grouped together. Variation can occur because of changes in the inventory of medical devices in, e.g., a hospital.

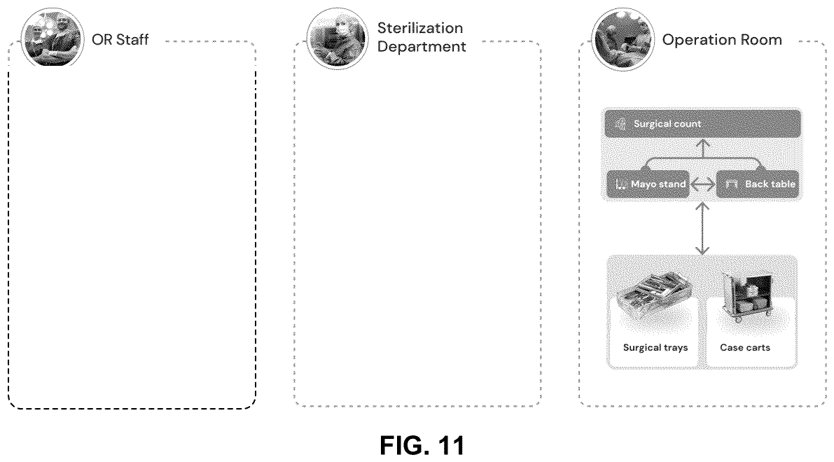

Moreover, FIG. 11 presents a drawing illustrating an example of a flow associated with the surgical procedure of FIG. 10, which is performed in an operating room (e.g., by a nurse or a surgical technician). Notably, a surgical count of medical devices may be obtained from a Mayo stand and/or a back table in an operating room.

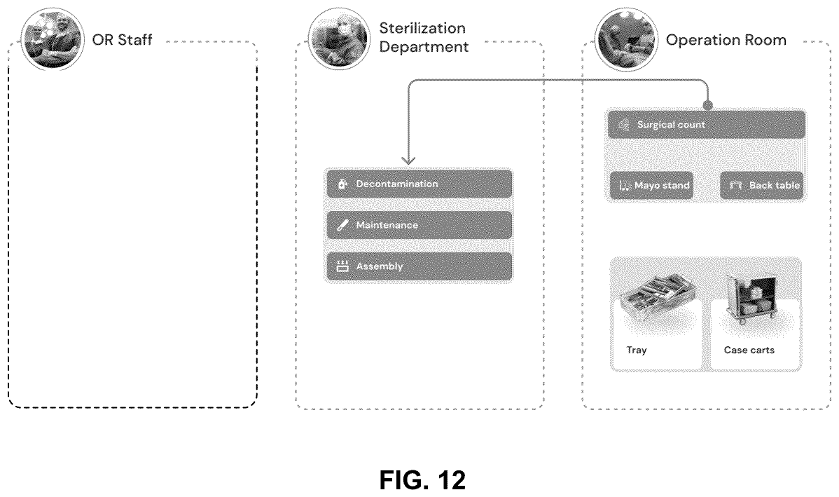

Then, as shown in FIG. 12, which presents a drawing illustrating an example of a flow associated with the surgical procedure of FIGS. 10 and 11, the sterilization department may decontaminate medical devices and/or perform maintenance on one or more medical devices, prior to assembly of trays, peel packs and/or case carts for use in a subsequent instance of a surgical procedure.

The flows shown in FIGS. 10-12 are largely manual and, as a consequence, are subject to error and waste. The disclosed monitoring and assembly techniques can address these inefficiencies and sources of error.

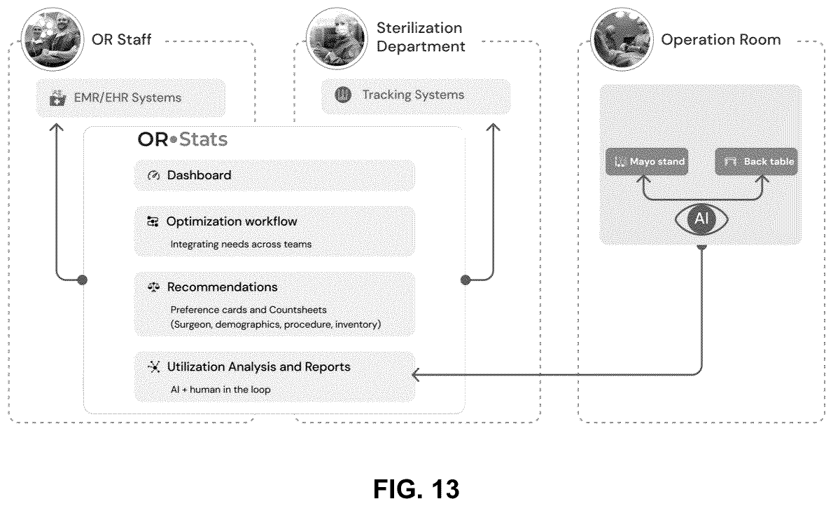

Notably, as shown in FIG. 13, which presents a drawing illustrating an example of a flow associated with a surgical procedure, the monitoring and assembly techniques may be provide a semi-automated or an automated wat for the operating-room staff and the sterilization departments to optimize workflow and generate recommendations (such as dynamic or data-driven preference cards and count sheets, which are different from existing static preference cards and count sheets) based at least in part on feedback from the operating room (such as usage of medical devices based at least in part on monitoring of a Mayo stand and/or a back table in an operating room, which may be used to generate utilization reports).

The monitoring techniques may: have a small physical footprint in the operating room; offer improved security; and/or no interruption to the operating-room staff. Moreover, the monitoring techniques may provide: accurate identification of millions of instruments; large-scale data analysis; insightful reports for different teams; and/or integrated workflows. For example, visual capture in the monitoring techniques may be performed using a cellular telephone or a network-enabled camera. Image sensor(s) in these electronic devices may be aimed (and, thus, their fields of view may encompass) a Mayo stand and/or back table(s) in an operating room. The disclosed monitoring techniques may enable data-based tray assembly, which may allow trays to be simplified.

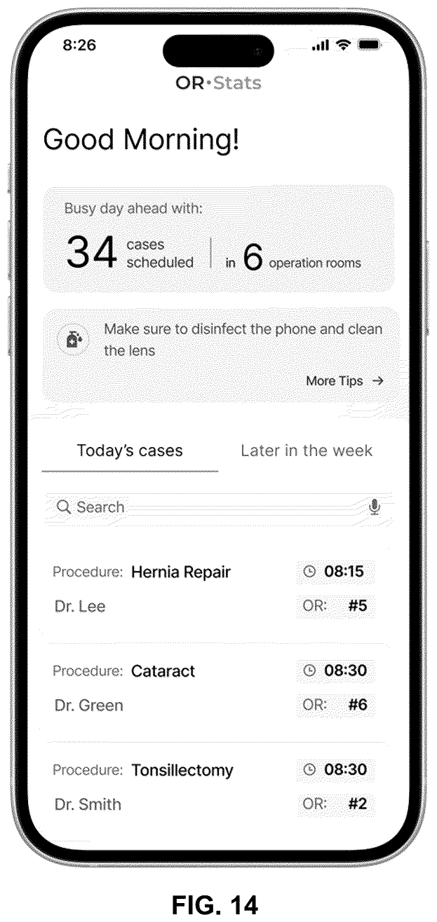

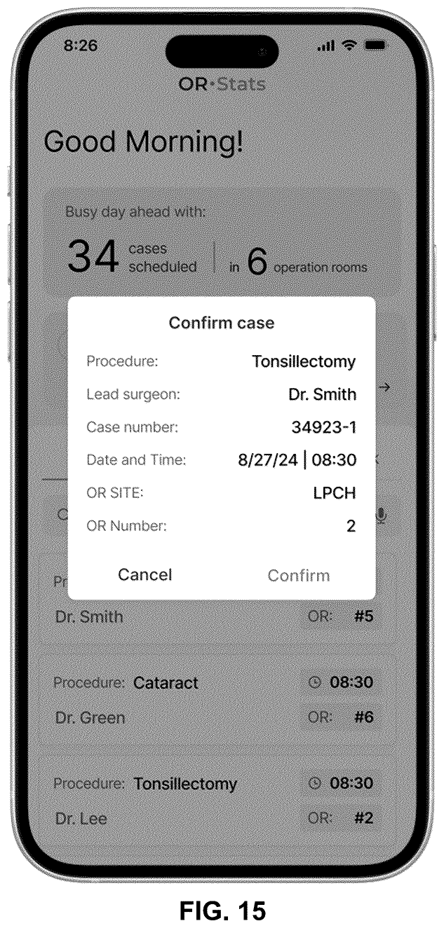

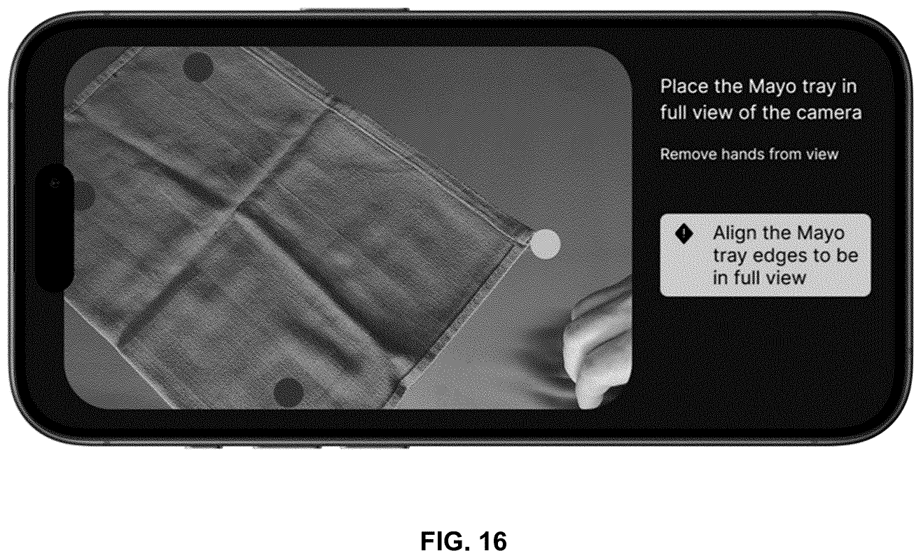

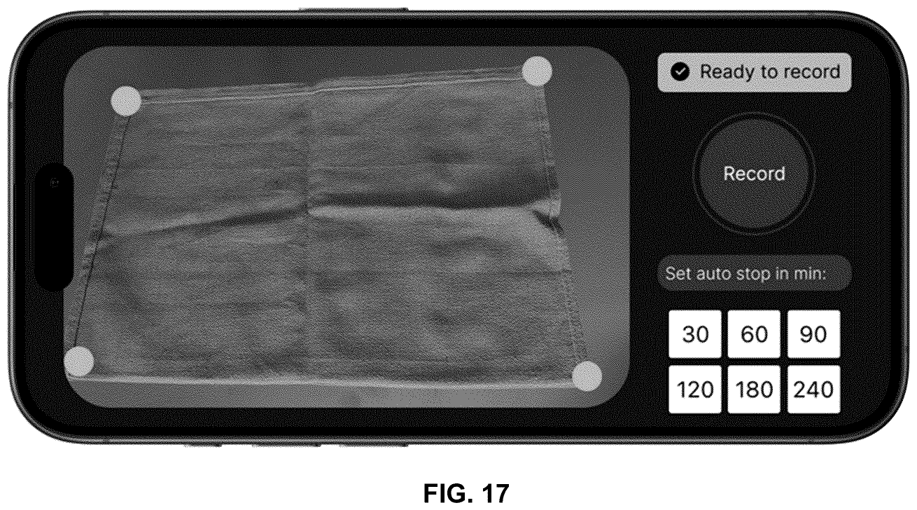

FIGS. 14 and 15 present drawings illustrating examples of user interfaces in the monitoring techniques, which may provide streamlined flow or use. Moreover, as shown in FIGS. 16 and 17, which presents drawings illustrating examples of user interfaces, the user interface may provide simple setup, which may guide a user (such as by provide instructions on where to point a camera or image sensor and, thus, what objects to include in its field of view) and then automatically verify their actions (e.g., by confirming that edges of the Mayo stand (such as the towel edges) are within the field of view).

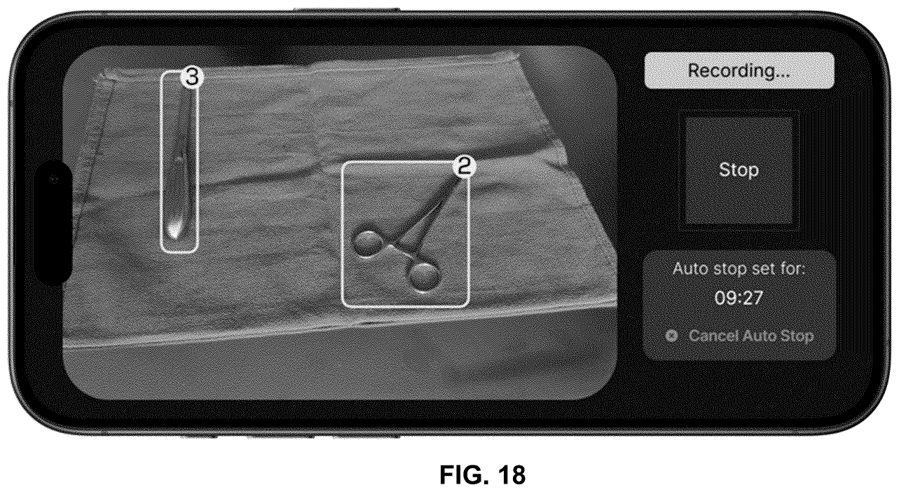

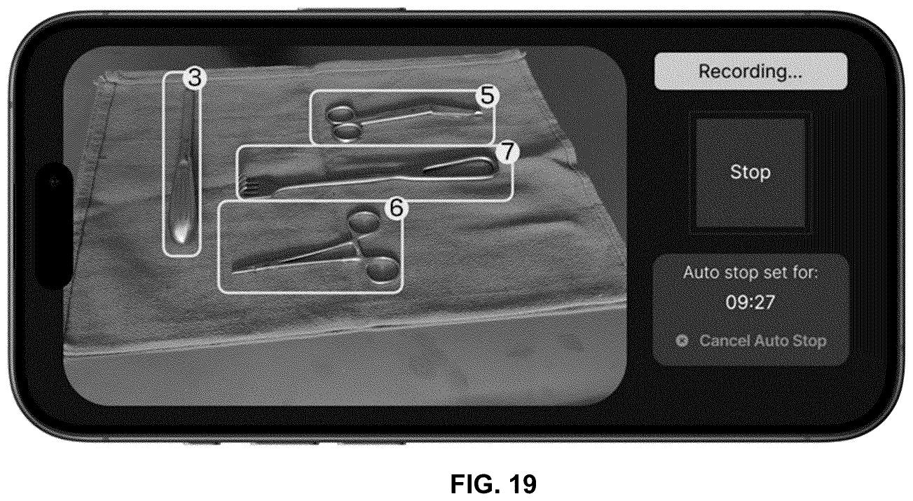

Then, as shown in FIGS. 18 and 19, which presents drawings illustrating examples of user interfaces, the user interface may provide hands-free (i.e., automated) recording. For example, the recording may be: timer-based, linked to a schedule, trigger-based or continuous. Note that during the monitoring, different medical devices may be identified. This may include the location and/or orientation of a given medical device in the field of view of an image sensor.

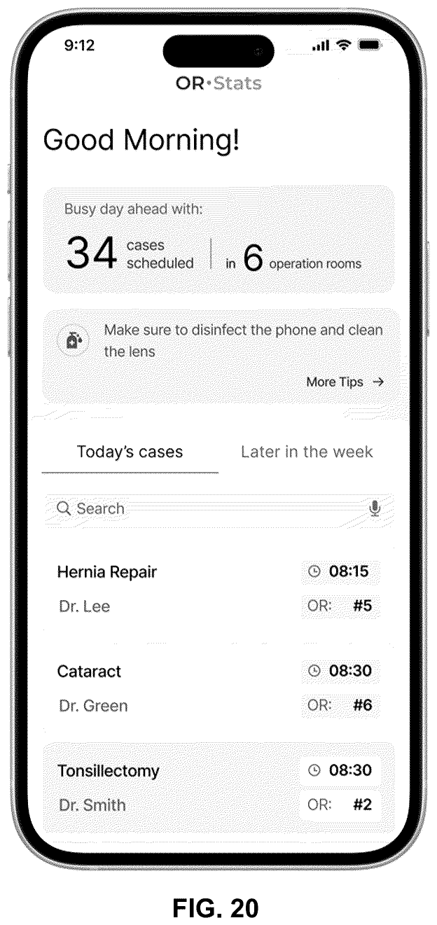

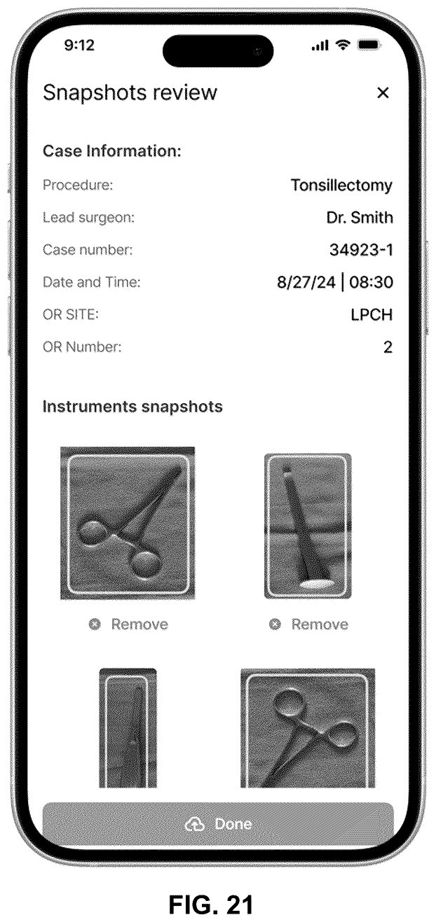

FIGS. 20 and 21 present drawings illustrating examples of user interfaces. Notably, the user interface may present: a schedule of medical procedures; and/or one or more identified medical devices (such as surgical instruments).

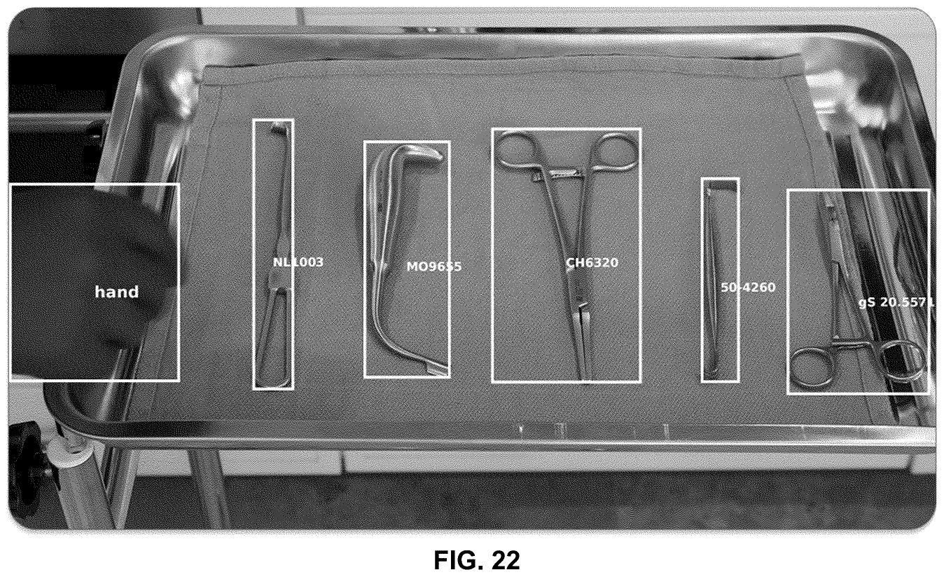

Moreover, as shown in FIG. 22, which presents a drawing illustrating an example of an image of a tray of medical devices during a surgical procedure, identified objects (such as a gloved hand or surgical devices) in one or more acquired images associated with a field of view of an image sensor may be highlighted. Note that, in the assembly techniques, the tray of medical devices may be assembled based at least in part on information about usage in a utilization report that is generated during the monitoring techniques. In some embodiments, the assembly techniques may involve multiple trays of medical devices, and there may be instances of a given medical device in more than one tray of medical devices.

In some embodiments, the electronic device (such as the cellular telephone or network-enabled camera, may automatically redact (such as removing or obfuscating) sensitive information (such as protected health information, text, faces, identities, barcodes, etc.).

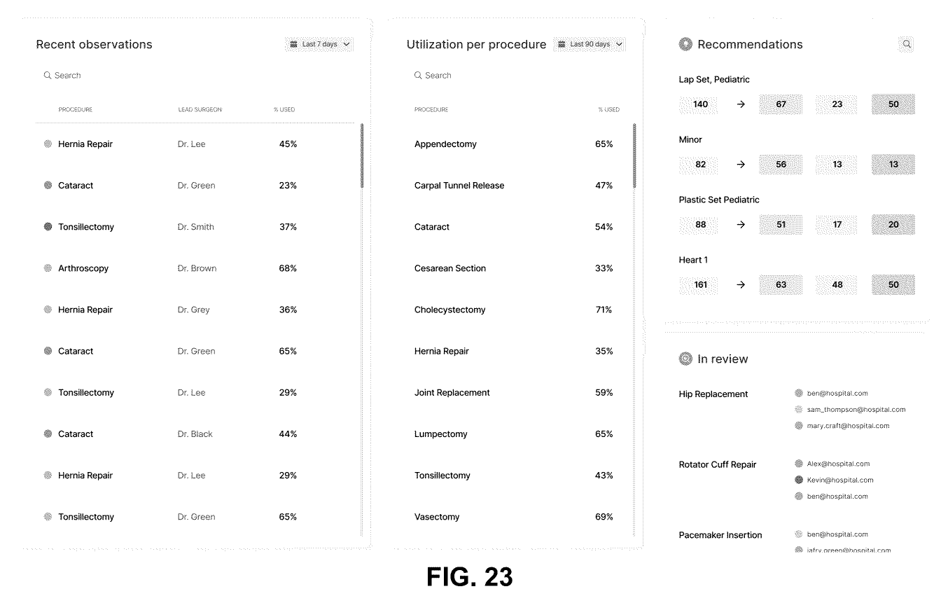

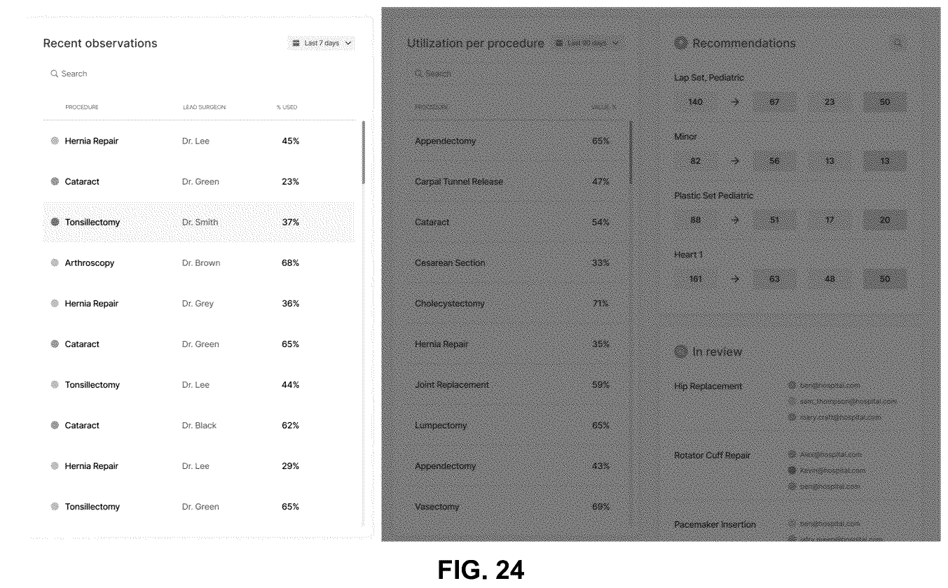

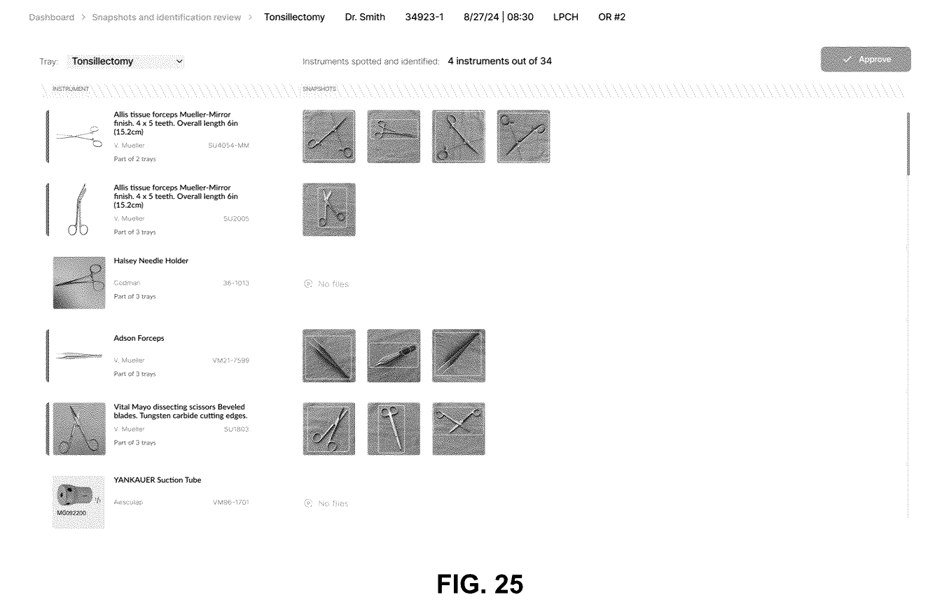

As shown in FIG. 23, which presents a drawing illustrating an example of a user interface, the electronic device and/or the computer system in the monitoring techniques may also provide a web-based dashboard. This may provide a centralized location for workflows, reports and/or recommendations. Moreover, as shown in FIGS. 24 and 25, which present drawings illustrating examples of user interfaces, the dashboard may provide a user (such as surgical technician) full control over the data. For example, snapshots of medical devices during a medical procedure (such as a surgical procedure) may be reviewed. This may provide accuracy at scale, and may allow improvement of one or more predictive models that are used in the monitoring techniques (such as retraining of at least a portion of one or more machine-learning models, e.g., one or more neural networks, and/or modification of one or more prompts used with the one or more neural networks).

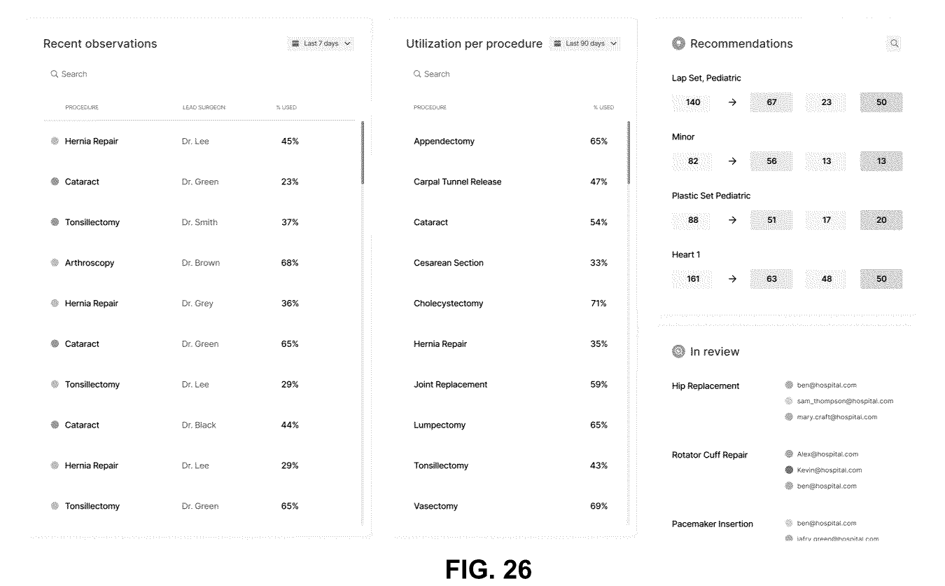

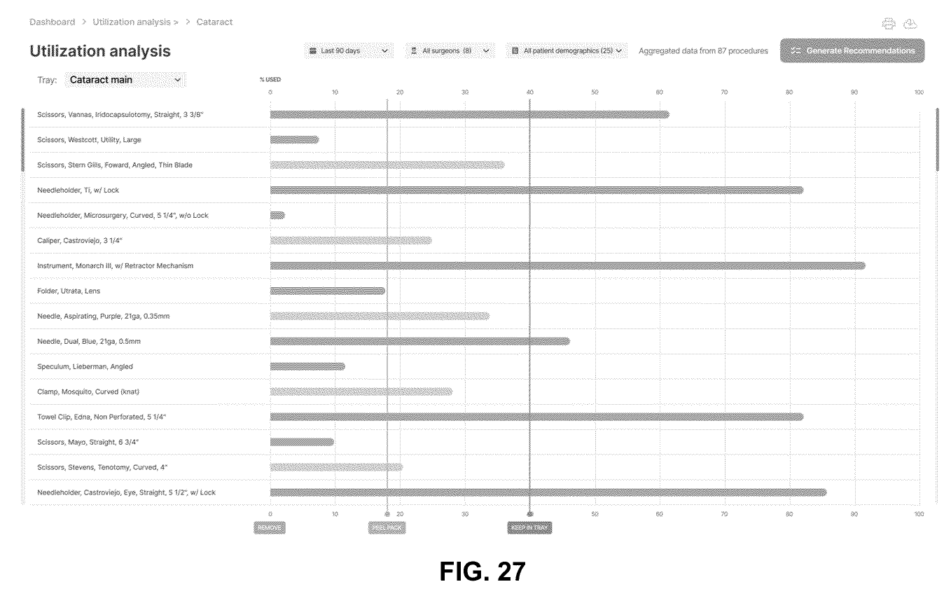

Moreover, as shown in FIGS. 26 and 27, which present drawings illustrating examples of user interfaces, the monitoring techniques may provide insights, such as: observations, utilization reports, and/or recommendations. This information may allow a user (such as surgical technician) to gain understanding about usage across procedures, surgeons, patient demographics, etc.

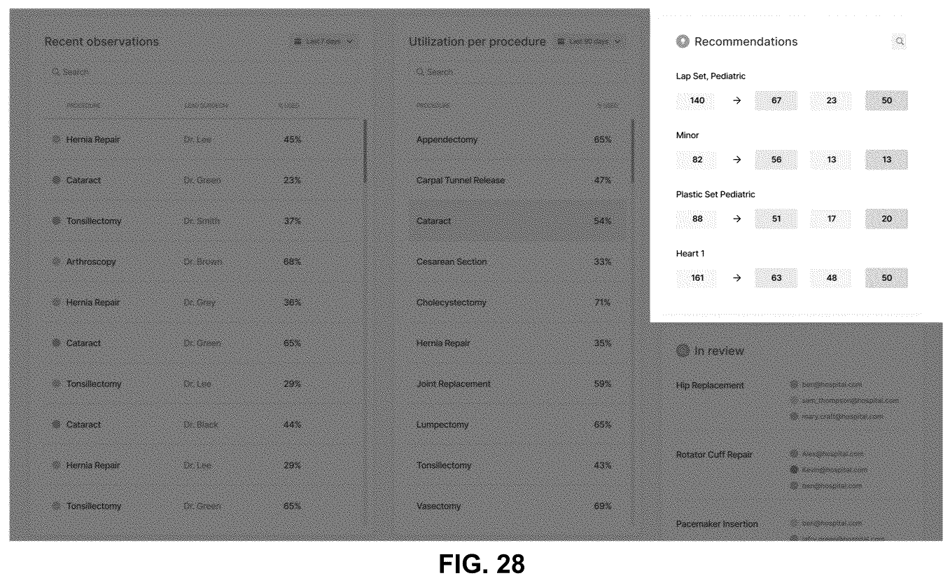

Furthermore, FIG. 28 presents a drawing illustrating an example of a user interface. This user interface may provide one or more data-driven recommendations, such as a recommendation based at least in part on a utilization report. In this way, the monitoring techniques may provide clear recommendations based at least in part on aggregated data at scale.

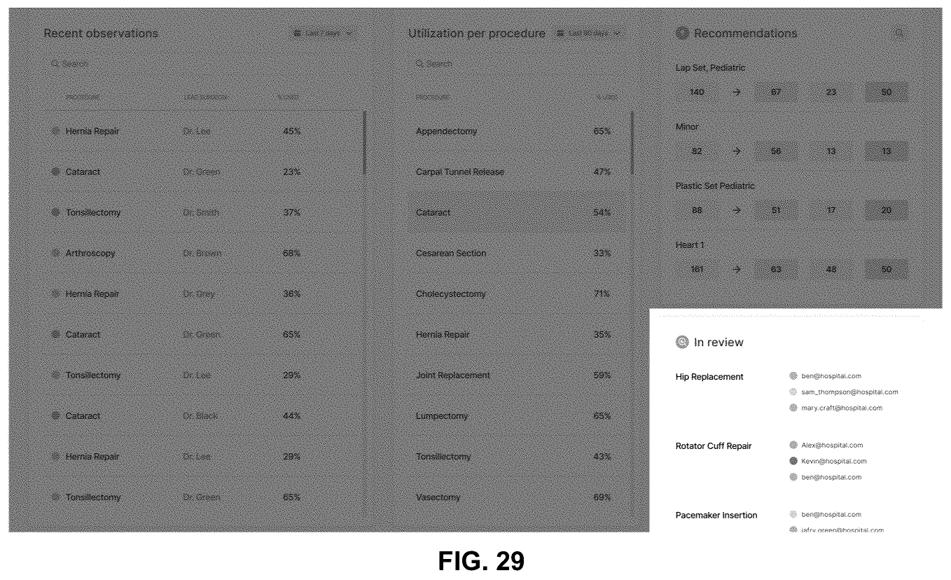

Additionally, FIG. 29 presents a drawing illustrating an example of a user interface. This user interface may provide information associated with a workflow. The information may be easy for users to follow, which may allow changes to be applied across teams.

A given user interface in the disclosed embodiments may include one or more additional features, one or more locations of one or more features may be changed, two or more features may be combined into a single feature, and/or a single feature may be divided into two or more features.

In some embodiments, the monitoring techniques and/or the assembly techniques may be performed separately or in conjunction with each other. The monitoring techniques and/or the assembly techniques may include: monitoring, optimization (which is sometimes referred to as ‘tray trimming’ or ‘preference card optimization’), and/or assembly. These operations may be performed sequentially, and there may be feedback from the assembly to the monitoring.

The monitoring may include monitoring the usage of medical devices and/or observing the surgical count (or surgical instruments) during one or more surgical procedures in one or more hospitals or surgical facilities. For example, a ‘portal’, such as a Mayo tray or a back table, may be observed using a camera. Because the portal may move, it may be tracked, e.g., using a pan tilt and zoom (PTZ) camera or by a human (in response to an alert when the portal is not in a field of view). In some embodiments, triggers, such as a hand leaving a frame, may be used to solve occlusion (e.g., when a hand is not present, a camera may have an unobstructed view in a portal of the surgical instruments and/or medical devices). The monitoring may, at least in part, be facilitated or overseen by a circulating nurse and/or a surgical technician. However, the monitoring of historical or usage information may occur with minimal support from operating-room staff.

In some embodiments, the triggers may include or may be used to provide implicit or explicit starting and stopping of monitoring (such as a footswitch toggle to start and stop; or a distinct hand gesture, e.g., “thumbs up”). Moreover, a trigger may be used to record the surgical count. For example, because a gloved hand places and removes surgical instruments, when a gloved hand exits the portal, images may be acquired. Gloved hands place and remove instruments. Thus, the gloved hand exiting the frame may be a ‘portal capture event’ of interest. Alternatively or additionally, a hand may signal that something placed on or in the portal came from the outside.

Moreover, the surgical instruments, surgical soft goods or medical devices in or on the portal may be autonomously identified, e.g., in the portal and without human involvement (beyond placing or holding items in the portal). For example, items may be identified by: performing a barcode scan (which may provide a ‘checkout experience’); and/or when items are placed on a tray 2 cm apart or in a particular orientation. In some embodiments, the identification may be approximate, such as narrowing down to possible surgical instruments. Alternatively or additionally, the imaging sensor and a detection system may autonomously acquire subsequent images focusing on key features (such as barcodes, device tips, and text) to facilitate subsequent identification. Note that items that were missing, e.g., from a case cart, may be tracked and fetched. Using the surgical count, usage data may be extracted, such as use of surgical soft goods.

Furthermore, sensitive information may be redacted during the monitoring. Note that the observed surgical count may be used to obtain surgical soft goods and single-use medical devices. For example, sensitive information may be redacted to comply with HIPAA. The redaction techniques may include: detecting faces, text, codes, badges, identification bands, etc., that may make their way into a frame; and/or acquiring images only of the portal and use explicit ‘triggers; to decide which frames are persisted for data collection. The detection of sensitive information can be done using an object detector trained on sensitive information using one or more of the disclosed identification techniques. Additionally, redaction may include: finding a bounding box or segmentation boundary that includes sensitive information and then making the corresponding pixels black; and/or skipping a frame that includes sensitive information.

Note that additional data may be combined with the image data from the portal. For example, the additional data may correspond to activity during the surgery or medical procedure. Notably, the additional data may include audio, and analysis of the audio and images may allow an activity during the surgery or medical procedure to be determine, such as what the surgical staff are counting (“Sponges, one, two, . . . ”). Thus, the additional data may include specific keywords that are used to determine the surgical count. Note that the monitoring may include a time of a surgical procedure (e.g., from computer system 120 in FIG. 1).

The monitoring may include determining or recognizing when the portal is in a good state for recording. For example, this may include: recognizing when there is an occlusion (such as at least a portion of a person blocking the portal); using an object detector or portal tracking to ensure the view of the portal is unobstructed and that the portal remains in the field of view (such as using computer-vision techniques to locate the portal in each frame of video); recognizing and taking action when the portal is out of the field of view (such as providing a pan-tilt-zoom command so that the portal fills the frame, or alerting a human when the view of the portal cannot be recovered autonomously); and/or triggering the acquisition of images and/or the additional data in or associated with the portal.

Furthermore, given data and balanced economic objectives, and based at least in part on historical usage information, the optimization may be used to recommend changes to count sheets, case carts, peel packs and/or preference cards for one or more future surgical procedures stored in a tracking system and/or an EMR system. For example, a note may be added to the preference card after a surgical procedure and scanned into a monitoring system. Note that trays may be audited and aligned with the recommended changes. In some embodiments, the optimization may, at least in part, be facilitated or overseen by operating room and/or sterile processing department staff.

The optimization may include: showing decision-makers or stakeholders (who may need to approve any recommended changes) supporting data in charts; making recommendations regarding count sheets and/or preference cards; and/or recommending moving some medical devices from trays to peel packs. Note that the optimization may not be performed ‘on the fly’ at assembly time. Instead, it may be preceded by an update to the tracking system and/or EMR system, and may require pre-approval by a stakeholder.

The information presented to the stakeholders may include: the medical device and soft good usage occurrence counts or percentages; and/or charts, tables, and other visualizations that include user-interface features (such as icons, objects, etc.) that facilitate a user viewing medical device usage by categorical factors, such as the surgical procedure, surgeon, patient information, etc. Note that, as needed, the presented information may include visualizations of certainty and confidence, such as color codes, error bars, support, and confidence intervals. In some embodiments, the presented information may include demographic information about the patient, the type of surgery, and the surgeon. This may allow better optimization of the tray to the case. The presented information may come from an EMR system, and the surgical instrument information comes from a tracking system.

Furthermore, a recommendation may be based at least in part on the historical information, such as: use of a medical device (e.g., a Vannas Iridocapsulotomy Straight 3⅜″ Scissors, a Westcott Large Utility Scissors, a Titanium Needleholder with Lock, a Microsurgery Curved 5¼″ Needleholder without Lock, etc.) in the last 90 days, by multiple surgeons, across multiple patient demographics, integrated from multiple surgical procedures, etc. A recommendation may include: removing a very rarely used medical device from a tray; removing a package of soft goods or using a smaller package with fewer items (because open packages are typically thrown out); providing instructions to not open a soft goods package until items are needed instead of during operating-room setup; changing the count of a medical device or soft goods (e.g., because fewer are needed than required in the count sheet); moving a medical device from a tray to a peel-pack, or keeping it in storage near the operating room but not on the case cart, and, thus, not having to reprocess it when it is unused; and/or adding a medical device or soft good to a tray or case cart because it will likely be needed. In order to support recommendations, stakeholders may provide unit economic data to one or more systems, such as: the cost when a medical device or soft goods is absent or when there are too few instances of an item; the cost of reprocessing a medical device; and/or the costs and reimbursement costs of soft goods. Because observations are often noisy, analytical techniques may be used to better model uncertainty and obtain better statistical estimates. For example, because identification is often approximate, medical device counts may be adjusted for bias on undercounting and overcounting. Alternatively or additionally, adjustments may be made for medical device equivalences (or medical devices that can be substituted for each other) and/or medical devices from different manufacturers may be grouped into equivalence groups. Because the observations may be sparse for a specific surgical procedure in a specific hospital, the data may be anonymized and pooled from similar surgical procedures across multiple hospitals. This may improve the confidence and accuracy of recommendations.

In some embodiments, one or more pretrained predictive models may be used to make recommendations by pooling or aggregating observations from the same surgical and/or medical procedure(s) across different hospitals and/or surgical centers. For example, the one or more pretrained predictive models may use Bayesian inference or a hierarchical Bayesian model based at least in part on variational inference or Markov chain Monte Carlo. In these Bayesian techniques, the prior probability may be from pooled data for surgical and/or medical procedure(s) and the posterior probability of the need for medical devices and/or surgical soft goods in a given surgical procedure may be from the case-cart observations for a particular surgical and/or medical procedure(s) in a particular hospital (which correspond to a case cart). This may allow a recommendation to be made after a few observations.

The analysis may also quantify the uncertainty in the recommendations in a data-efficient manner, giving cost estimates with certainty estimates (e.g., a credibility interval) on case cart configurations and expected cost savings. Consequently, the Statistical models may incorporate the unit economic data provided by stakeholders. Given statistical models and cost estimates, the optimization procedure may make recommendations. For example, a binary decision variable may be defined for including a medical device in a count sheet and the optimization problem may be solved using an optimization technique, such as: greedy heuristics, integer linear programming, and/or dynamic programming. Note that with recommendations supported by data and a defensible analysis of future cost savings, stakeholders can approve and implement recommended changes to preference cards and count sheets. Note that any updates to tray count sheets and/or preferred cards may be implemented outside the disclosed techniques. Instead, the disclosed techniques may offer integration and recommendations to facilitate such updates, e.g., by giving the medical team control over the data and decisions.

Additionally, building a case cart and assembling trays may include: performing tray assembly and/or preparing case carts based at least in part on previously optimized information from the tracking system and/or the EMR system. Alternatively, the optimization may be performed dynamically or on-the-fly during the assembly and/or during surgical procedures. Tray assembly may be performed on the clean-side, post decontamination and pre-sterilization, while case carts may be built post sterilization. For example, the assembly may be performed by sterile processing department staff, such as when a sterile processing technician performs an inventory check and inspection of a tray against the medical devices listed on a count sheet (which may have been previously optimized).

Note that assembly may include matching based at least in part on one or more count sheets that list medical devices by name, brand name, and catalog number given by the hospital. This may involve matching the medical devices on a count sheet to information in one or more data structures. In some embodiments, the matching may involve: applying lexical transformations of the catalog numbers, such as the case, punctuation, and spacing, to achieve accurate matching without introducing false positives; checking alternative spellings of the brand names and alternative branding caused by rebranding and white-labeling; and/or disambiguating and verifying an association or a link based at least in part on a thresholded semantic similarity check of the medical device name. In some embodiments, the techniques may provide a list of medical device alternatives that may be substituted for a medical device listed on the count sheet. For example, a recommendation in the optimization may be for an equivalent surgical instrument or medical device that may be provided during the assembly, such as when there is a difference between the surgical count (or the actual contents of a tray) and a count sheet (or a bill of materials). Thus, in the disclosed techniques, a count sheet may include a list of medical devices and their equivalents. An equivalent device may be inferred from: an alternative to a medical device, which may be explicitly mentioned on the count sheet; visual equivalence (such as medical devices for which the cosine similarity of their embedding vectors is less than a predefined value); functional equivalence (such as by thresholding a distinct metric for one or more properties that reflect the function of a medical device in a knowledge graph, e.g., a type hierarchy, dimensions, and/or physical properties); SKU equivalence (such as medical devices from the same manufacturer that are visually equivalent, but which may different in quality); vendor equivalent (such as the same medical device under different branding); and/or usage equivalence (e.g., medical device A was assembled as medical device B for many count sheets).

For example, for visual equivalence, suppose X, Y, and Z are medical devices, where X is equivalent to Y, but X is not equivalent to Z. Let X′, Y′, and Z′ be representations of X, Y, and Z, respectively. Notably, X′ may be a JPEG image of surgical scissors. Alternatively, X′ may be a description of a forceps natural language. In some embodiments, X′ is a Resource Description Framework (RDF) description based at least in part on an ontology. The first representation can test for visual equivalence, whereas the second can test for functional equivalence for sufficiently good descriptions. Moreover, let F be a function that maps F(X′) (which is sometimes referred to as an ‘encoder’ or ‘embedding function’) onto a d-dimensional hypersphere (e.g., d equal to 512) found by solving the optimization problem that F(X′) is closer to F(Y′) than F(Z′). Then, there exists an angle, θ, between 0 and π radians found over a sample to best achieve a particular metric, such as the balance of false positives and false negatives, e.g., an F1 score, which is the harmonic mean of precision and recall. Note that equivalence may be reflexive and symmetric, but may not be transitive. Consequently, technically we are talking about approximate equivalence based at least in part on similarity. Moreover, the preceding definition and discussion also holds for functional equivalence, albeit with a different encoder.

Note that the disclosed identification techniques may be used during assembly. For example, once the medical devices on the count sheet are linked, the electronic device may use the set of medical devices (which may include the count sheet and other information, such as images) to identify the medical device using the identification techniques or determine that a given medical device does not belong to a tray. Alternatively, after identifying a medical device or an equivalent, the electronic device may solicit approval from a surgical technician to update the count on the corresponding row of the count sheet, which a tracking system may manage. This is sometimes referred to as ‘marked as assembled.’ In some embodiments, the assembly may include guided and verified inspection. Notably, once a medical device has been identified and accepted, a surgical technician may be presented with inspection instructions to ensure that the medical device is fit for use, such as checking the sharpness of scissors. Inspection may involve the use of a camera and computer vision to detect visual abnormalities, such as scratches, pitting, corrosion, etc.

In some embodiments, a robot may assist with inspection and identification during the assembly, but the robot may or may not perform the assembly. Thus, the assembly may be semi-automatic (e.g., performed jointly with a robot) or autonomous (e.g., performed solely by a robot). For example, while the electronic device may assist a sterile processing technician in performing tray assembly, in other embodiments, such as when the identification and inspection problems are solved, automated assembly may occur. Notably, a robot may use a computer-vision model and/or a depth camera (e.g., based at least in part on time of flight, structured light and/or stereoscopic vision) to identify a grab point (such as 3D coordinates) on a medical device in a bin. The grab point 3D location may be sent to a robot arm. The robot arm may attempt to pick up the medical device at the grab point. It may shake the medical device to separate it from other medical devices in a pile. This process may be repeated until a medical device is separated from other medical devices in a bin. Note that the robot may have a grab pose prediction model to determine the best gripper, arm path, and pose to grab the medical device at the grab point. Moreover, the robot may generate a motion plan to position the robot at the grab point. After grabbing the medical device, the robot may place it in a portal for identification. For example, when the portal is on a glass surface, the robot may use two cameras (above and below a surface of the portal), to identify the medical device. Once again, motion planning may be used to place the medical device in a desired pose in the portal. This may involve the robot placing and picking the medical device back up at a more centered grab point in order to achieve a desired position and orientation in the portal. Furthermore, after identification, the robot may place the medical device in a location and orientation specified by the count sheet, such as a row order. Alternatively or additionally, the robot may: snap medical devices into fixtured positions on a tray for robotic assembly; place medical devices onto a belt or magnetically levitated tiles for sorting surgical instruments and another robot arm may place them onto a tray or into a tray fixture; and/or provide the sorted medical devices to a surgical technician, who places the medical devices on or into a tray. Then, the electronic device may autonomously mark the count sheet row as complete.

We now describe embodiments of an electronic device, which may perform at least some of the operations in the monitoring techniques and/or assembly techniques. FIG. 30 presents a block diagram illustrating an example of an electronic device 3000, such as access point 112, base station 116, computer system 120 and/or one of electronic devices 110-1. This electronic device includes processing subsystem 3010, memory subsystem 3012, and networking subsystem 3014. Processing subsystem 3010 includes one or more devices configured to perform computational operations. For example, processing subsystem 3010 can include one or more microprocessors, ASICs, microcontrollers, programmable-logic devices, one or more graphics process units (GPUs) and/or one or more digital signal processors (DSPs).

Memory subsystem 3012 includes one or more devices for storing data and/or instructions for processing subsystem 3010 and networking subsystem 3014. For example, memory subsystem 3012 can include dynamic random access memory (DRAM), static random access memory (SRAM), and/or other types of memory. In some embodiments, instructions for processing subsystem 3010 in memory subsystem 3012 include: one or more program modules or sets of instructions (such as program instructions 3022 or operating system 3024), which may be executed by processing subsystem 3010. Note that the one or more computer programs may constitute a computer-program mechanism. Moreover, instructions in the various modules in memory subsystem 3012 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 3010.

In addition, memory subsystem 3012 can include mechanisms for controlling access to the memory. In some embodiments, memory subsystem 3012 includes a memory hierarchy that comprises one or more caches coupled to a memory in electronic device 3000. In some of these embodiments, one or more of the caches is located in processing subsystem 3010.

In some embodiments, memory subsystem 3012 is coupled to one or more high-capacity mass-storage devices (not shown). For example, memory subsystem 3012 can be coupled to a magnetic or optical drive, a solid-state drive, or another type of mass-storage device. In these embodiments, memory subsystem 3012 can be used by electronic device 3000 as fast-access storage for often-used data, while the mass-storage device is used to store less frequently used data.

Networking subsystem 3014 includes one or more devices configured to couple to and communicate on a wired and/or wireless network (i.e., to perform network operations), including: control logic 3016, an interface circuit 3018 and one or more antennas 3020 (or antenna elements). (While FIG. 30 includes one or more antennas 3020, in some embodiments electronic device 3000 includes one or more nodes, such as nodes 3008, e.g., a network node that can be connected or coupled to a network, a connector or a pad that can be coupled to the one or more antennas 3020. Thus, electronic device 3000 may or may not include the one or more antennas 3020.) For example, networking subsystem 3014 can include a Bluetooth™ networking system, a cellular networking system (e.g., a 3G/4G/5G network such as UMTS, LTE, etc.), a universal serial bus (USB) networking system, a networking system based on the standards described in IEEE 802.11 (e.g., a Wi-Fi® networking system), an Ethernet networking system, a cable modem networking system, and/or another networking system.

Networking subsystem 3014 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ or a ‘connection’ between the electronic devices does not yet exist. Therefore, electronic device 3000 may use the mechanisms in networking subsystem 3014 for performing simple wireless communication between the electronic devices, e.g., transmitting advertising or beacon frames and/or scanning for advertising frames transmitted by other electronic devices as described previously.

Within electronic device 3000, processing subsystem 3010, memory subsystem 3012, and networking subsystem 3014 are coupled together using bus 3028. Bus 3028 may include an electrical, optical, and/or electro-optical connection that the subsystems can use to communicate commands and data among one another. Although only one bus 3028 is shown for clarity, different embodiments can include a different number or configuration of electrical, optical, and/or electro-optical connections among the subsystems.

In some embodiments, electronic device 3000 includes a display subsystem 3026 for displaying information on a display, which may include a display driver and the display, such as a liquid-crystal display, a multi-touch touchscreen, etc.

Electronic device 3000 can be (or can be included in) any electronic device with at least one network interface. For example, electronic device 3000 can be (or can be included in): a desktop computer, a laptop computer, a subnotebook/netbook, a server, a tablet computer, a smartphone, a cellular telephone, a smartwatch, a consumer-electronic device, a portable computing device, a drone, a headset (such as an augmented-reality headset or a virtual-reality headset), a camera (such as a security camera), a camera coupled with deep learning, a depth-sensitive camera (such as a stereoscopic camera, a time-of-flight camera, a camera that uses structured light, etc.), an infrared camera, a smart speaker, a smart doorbell (which may include a camera or an image sensor), smart glasses, a robot, an access point, a transceiver, a router, a switch, communication equipment, a base station, a controller, test equipment, and/or another electronic device.

Although specific components are used to describe electronic device 3000, in alternative embodiments, different components and/or subsystems may be present in electronic device 3000. For example, electronic device 3000 may include one or more additional processing subsystems, memory subsystems, networking subsystems, and/or display subsystems. For example, electronic device 3000 may include one or more sensors and/or measurement devices in a measurement subsystem 3030. Additionally, one or more of the subsystems may not be present in electronic device 3000. Moreover, in some embodiments, electronic device 3000 may include one or more additional subsystems that are not shown in FIG. 30. Also, although separate subsystems are shown in FIG. 30, in some embodiments some or all of a given subsystem or component can be integrated into one or more of the other subsystems or component(s) in electronic device 3000. For example, in some embodiments program instructions 3022 are included in operating system 3024 and/or control logic 3016 is included in interface circuit 3018.

Moreover, the circuits and components in electronic device 3000 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit (which is sometimes referred to as a ‘communication circuit’) may implement some or all of the functionality of networking subsystem 3014 (or, more generally, of electronic device 3000). The integrated circuit may include hardware and/or software mechanisms that are used for transmitting wireless signals from electronic device 3000 and receiving signals at electronic device 3000 from other electronic devices. Aside from the mechanisms herein described, radios are generally known in the art and hence are not described in detail. In general, networking subsystem 3014 and/or the integrated circuit can include any number of radios. Note that the radios in multiple-radio embodiments function in a similar way to the described single-radio embodiments.

In some embodiments, networking subsystem 3014 and/or the integrated circuit include a configuration mechanism (such as one or more hardware and/or software mechanisms) that configures the radio(s) to transmit and/or receive on a given communication channel (e.g., a given carrier frequency). For example, in some embodiments, the configuration mechanism can be used to switch the radio from monitoring and/or transmitting on a given communication channel to monitoring and/or transmitting on a different communication channel. (Note that ‘monitoring’ as used herein comprises receiving signals from other electronic devices and possibly performing one or more processing operations on the received signals)

In some embodiments, an output of a process for designing the integrated circuit, or a portion of the integrated circuit, which includes one or more of the circuits described herein may be a computer-readable medium such as, for example, a magnetic tape or an optical or magnetic disk. The computer-readable medium may be encoded with data structures or other information describing circuitry that may be physically instantiated as the integrated circuit or the portion of the integrated circuit. Although various formats may be used for such encoding, these data structures are commonly written in: Caltech Intermediate Format (CIF), Calma GDS II Stream Format (GDSII), Electronic Design Interchange Format (EDIF), OpenAccess (OA), or Open Artwork System Interchange Standard (OASIS). Those of skill in the art of integrated circuit design can develop such data structures from schematics of the type detailed above and the corresponding descriptions and encode the data structures on the computer-readable medium. Those of skill in the art of integrated circuit fabrication can use such encoded data to fabricate integrated circuits that include one or more of the circuits described herein.

While the preceding discussion used an Ethernet, a Wi-Fi communication protocol and/or a cellular-telephone communication protocol as an illustrative example, in other embodiments a wide variety of communication protocols and, more generally, wired and/or wireless communication techniques may be used. Thus, the monitoring techniques and/or the assembly techniques may be used with a variety of network interfaces. Furthermore, while some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the monitoring techniques and/or the assembly techniques may be implemented using program instructions 3022, operating system 3024 (such as a driver for interface circuit 3018) or in firmware in interface circuit 3018. Alternatively or additionally, at least some of the operations in the monitoring techniques and/or the assembly techniques may be implemented in a physical layer, such as hardware in interface circuit 3018.

In some embodiments, wireless communication between the electronic device and the computer uses one or more bands of frequencies, such as: 900 MHz, 2.4 GHz, 5 GHZ, 6 GHz, 60 GHz, the Citizens Broadband Radio Spectrum or CBRS (e.g., a frequency band near 3.5 GHz), and/or a band of frequencies used by LTE or another cellular-telephone communication protocol or a data communication protocol. Note that the communication between electronic devices may use multi-user transmission (such as orthogonal frequency division multiple access or OFDMA).

Moreover, while the monitoring techniques and/or the assembly techniques were illustrated using one or more images, in other embodiments a wide variety of sensor or measurement inputs may be used, such as: an RF identifier, a weight, audio-based recognition and localization, 3D point clouds, etc.

While the preceding discussion illustrated the monitoring techniques and/or the assembly techniques using a particular application, in other embodiments the monitoring techniques and/or the assembly techniques may be used with an augmented-reality or a non-augmented reality application. For example, the monitoring techniques and/or the assembly techniques may be used in a batch-style use-case, in which a video feed is captured or streamed for analysis (i.e., an off-line use case, as opposed to a real-time use case). In these embodiments, the video may be processed frame-by-frame, and events may be ordered according to timestamps. Note that an event may be moving a medical device in the field of view, adding a medical device to the field of view, removing a medical device from the field of view, or changing the state of a medical device. Moreover, a report may be triggered that summarizes the events. For example, in a medical application, where an image sensor observes one or more medical devices, an event may be triggered each time a medical device is removed from the field of view, is used, and/or is added to the tray. The resulting report may summarize when and which medical devices were used.

Note that the batch-style use-case may also be used in a real-time monitoring or statistics mode. Notably, instead of sending a report at the end, the application may provide an alert while processing frames from the image sensor in real-time. This approach may be used, e.g., in in an operating room during surgery, such as when an image sensor detects that the wrong instrument has been placed on a tray, thereby potentially avoiding errors during surgery. Moreover, this approach may be used to automate the ‘Operating Room count,’ in which all medical devices and surgical supplies are counted before and after surgery to prevent medical devices and supplies from being left in the patient. For example, an image of a medical device in a predefined location may be compared to a count sheet (e.g., using optical character recognition) to automatically update the count during a medical procedure.

In the preceding description, we refer to ‘some embodiments.’ Note that ‘some embodiments’ describes a subset of all of the possible embodiments, but does not always specify the same subset of embodiments. Moreover, note that numerical values in the preceding embodiments are illustrative examples of some embodiments. In other embodiments of the monitoring techniques and/or the assembly techniques, different numerical values may be used.

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

1. An electronic device, comprising:
an interface circuit configured to communicate with a computer system;
an image sensor configured to acquire one or more images;
a processor, coupled to the interface circuit, the image sensor and a memory, configured to execute the program instructions; and
the memory configured to store the program instructions, wherein, when executed by the processor, the program instructions cause the electronic device to perform operations comprising:
monitoring use of medical devices in a tray of medical devices or a peel pack of medical devices during at least a medical procedure; and
providing, addressed to the computer system, a utilization report based at least in part on the monitoring, wherein the utilization report comprises historical usage of medical devices: in a type of the medical procedure, for a type of patient and/or by a given medical provider.

2. The electronic device of claim 1, wherein the medical procedure comprises a type of surgery and a given medical provider comprises a surgeon.

3. The electronic device of claim 1, wherein the operations comprise providing a recommendation for one or more medical devices to be included in another instance of the tray of medical devices or the peel pack of medical devices for use in a future medical procedure; and
wherein the recommendation is based at least in part on the historical usage of medical devices: in the type of the medical procedure, for the type of patient and/or by the given medical provider.

4. The electronic device of claim 1, wherein the recommendation is based at least in part on a preference of the given medical provider.

5. The electronic device of claim 1, wherein the monitoring comprises monitoring of a Mayo stand or a table holding at least the tray of medical devices or the peel pack of medical devices during the medical procedure.

6. The electronic device of claim 1, wherein the operations comprise predicting a medical device will be needed in the medical procedure, wherein the medical device is not included in the tray of medical devices or the peel pack of medical devices; and
providing an instruction to obtain the medical device or automatically obtaining the medical device.

7. The electronic device of claim 1, wherein the recommendation is provided to the computer system.

8. The electronic device of claim 1, wherein the computer system implements electronic medical record (EMR) software.

9. The electronic device of claim 1, the medical devices comprise: a surgical instrument, a surgical screw, surgical supplies or a surgical machine.

10. The electronic device of claim 1, wherein the monitoring comprises determining:
a type of a medical device in the medical devices; a classification of the medical device; an identification code of the medical device; membership of the medical device in a group; or a manufacturer of the medical device.

11. The electronic device of claim 10, wherein the group comprises a surgical tray, a peel pack or a set.

12. The electronic device of claim 1, wherein the type of patient has a type of medical condition, a severity of the type of medical condition, or both.

13. The electronic device of claim 1, wherein the operations comprise documenting a particular or discrete moment in the medical procedure.

14. The electronic device of claim 1, wherein the documentation is based at least in part on a trigger.

15. The electronic device of claim 14, wherein the trigger comprises: motion of a gloved hand; an absence of the gloved hand in a field of view of the image sensor; or placement of a medical device in a predefined portion in a frame of the image sensor.

16. The electronic device of claim 1, wherein the operations comprise redacting information acquired during the monitoring.

17. The electronic device of claim 1, wherein, during the monitoring, the operations comprise providing an instruction to change a perspective of the image sensor during the medical procedure so that an area of interest is include a field of view of the image sensor during the monitoring.

18. The electronic device of claim 1, wherein the monitoring comprises: identifying the medical devices observed during the monitoring; and verifying the medical devices observed during the monitoring.

19. A non-transitory computer-readable storage medium for use in conjunction with an electronic device, the computer-readable storage medium storing program instructions that, when executed by the electronic device, cause the electronic device to perform operations comprising:
monitoring use of medical devices in a tray of medical devices or a peel pack of medical devices during at least a medical procedure; and
providing, addressed to a computer system, a utilization report based at least in part on the monitoring, wherein the utilization report comprises historical usage of medical devices: in a type of the medical procedure, for a type of patient and/or by a given medical provider.

20. A method for monitoring usage of medical devices, comprising:
by an electronic device:
monitoring the usage of medical devices in a tray of medical devices or a peel pack of medical devices during at least a medical procedure; and
providing, addressed to a computer system, a utilization report based at least in part on the monitoring, wherein the utilization report comprises historical usage of medical devices: in a type of the medical procedure, for a type of patient and/or by a given medical provider.
